SEQUENCE LISTING (32 sequences)

SEQ ID NO: 1 - DNA, 20 bp, Artificial Sequence
  Description of Artificial Sequence Synthetic primer
cagccacattgggactgaga

SEQ ID NO: 2 - DNA, 20 bp, Artificial Sequence
  Description of Artificial Sequence Synthetic primer
cacacatggcattgctggat

SEQ ID NO: 3 - DNA, 25 bp, Artificial Sequence
  Description of Artificial Sequence Synthetic primer
cacttgaagagctattactggaggg

SEQ ID NO: 4 - DNA, 18 bp, Artificial Sequence
  Description of Artificial Sequence Synthetic primer
ctcggaccaccatccagg

SEQ ID NO: 5 - DNA, 20 bp, Artificial Sequence
  Description of Artificial Sequence Synthetic primer
ctgccacggcacagtcattg

SEQ ID NO: 6 - DNA, 20 bp, Artificial Sequence
  Description of Artificial Sequence Synthetic primer
tgcatcctttttcgccttgc

SEQ ID NO: 7 - DNA, 23 bp, Artificial Sequence
  Description of Artificial Sequence Synthetic primer
atttgaattccctgggtgagaag

SEQ ID NO: 8 - DNA, 22 bp, Artificial Sequence
  Description of Artificial Sequence Synthetic primer
cacaggggagaaatcgatgaca

SEQ ID NO: 9 - DNA, 30 bp, Artificial Sequence
  Description of Artificial Sequence Synthetic primer
acagaatatcaaccaacaagtgatattctc

SEQ ID NO: 10 - DNA, 21 bp, Artificial Sequence
  Description of Artificial Sequence Synthetic primer
gattctttcctttgaggccca

SEQ ID NO: 11 - DNA, 22 bp, Artificial Sequence
  Description of Artificial Sequence Synthetic primer
gacgtctttgatgatgaccctg

SEQ ID NO: 12 - DNA, 22 bp, Artificial Sequence
  Description of Artificial Sequence Synthetic primer
tgtgattctgaagtgctgcgtt

SEQ ID NO: 13 - DNA, 20 bp, Artificial Sequence
  Description of Artificial Sequence Synthetic primer
tctttgttcgaatccagcgc

SEQ ID NO: 14 - DNA, 22 bp, Artificial Sequence
  Description of Artificial Sequence Synthetic primer
ggaacgcacctttctggttaca

SEQ ID NO: 15 - DNA, 22 bp, Artificial Sequence
  Description of Artificial Sequence Synthetic primer
gcattttgaacgaggtcacagg

SEQ ID NO: 16 - DNA, 21 bp, Artificial Sequence
  Description of Artificial Sequence Synthetic primer
tatgcgaagcaccttggaagc

SEQ ID NO: 17 - DNA, 26 bp, Artificial Sequence
  Description of Artificial Sequence Synthetic primer
tcaggaaatttgcctattgaaaattt

SEQ ID NO: 18 - DNA, 31 bp, Artificial Sequence
  Description of Artificial Sequence Synthetic primer
gctttgtctttcttgttatcttttaagttgt

SEQ ID NO: 19 - DNA, 21 bp, Artificial Sequence
  Description of Artificial Sequence Synthetic primer
gtctttgacgctcggaactgt

SEQ ID NO: 20 - DNA, 19 bp, Artificial Sequence
  Description of Artificial Sequence Synthetic primer
gatggccgacctgatgttg

SEQ ID NO: 21 - DNA, 21 bp, Artificial Sequence
  Description of Artificial Sequence Synthetic primer
cttgggatccacactctccag

SEQ ID NO: 22 - DNA, 24 bp, Artificial Sequence
  Description of Artificial Sequence Synthetic primer
tttttggctaaacgctttcattaa

SEQ ID NO: 23 - DNA, 23 bp, Artificial Sequence
  Description of Artificial Sequence Synthetic primer
gctgacctgctggattacattaa

SEQ ID NO: 24 - DNA, 28 bp, Artificial Sequence
  Description of Artificial Sequence Synthetic primer
tgatcattacagtagctcttcagtctga

SEQ ID NO: 25 - DNA, 20 bp, Artificial Sequence
  Description of Artificial Sequence Synthetic primer
tgtcagtgcctgcagaccat

SEQ ID NO: 26 - DNA, 21 bp, Artificial Sequence
  Description of Artificial Sequence Synthetic primer
gctatgacttcggtttgggtg

SEQ ID NO: 27 - DNA, 18 bp, Artificial Sequence
  Description of Artificial Sequence Synthetic primer
tggctcagccagatgcag

SEQ ID NO: 28 - DNA, 24 bp, Artificial Sequence
  Description of Artificial Sequence Synthetic primer
ggtgatcctcttgtagctctccag

SEQ ID NO: 29 - DNA, 24 bp, Artificial Sequence
  Description of Artificial Sequence Synthetic primer
gagtgacaaacacgactgcaagat

SEQ ID NO: 30 - DNA, 21 bp, Artificial Sequence
  Description of Artificial Sequence Synthetic primer
ctgctttgcctacctctccct

SEQ ID NO: 31 - DNA, 25 bp, Artificial Sequence
  Description of Artificial Sequence Synthetic primer
gaccctcacactcagatcatcttct

SEQ ID NO: 32 - DNA, 21 bp, Artificial Sequence
  Description of Artificial Sequence Synthetic primer
ccacttggtggtttgctacga

(19) United States
(12) Patent Application Publication
(10) Pub. No.: US 2007/0042004 A1
(43) Pub. Date: Feb. 22, 2007

(54) Methods of Treating A Bacterial Infection using F. Tularensis Lipopolysaccharide

(76) Inventors: Stefanie Vogel, Columbia, MD (US); Leah E. Cole, Columbia, MD (US)

Correspondence Address: CASTELLANO MALM PLLC, 1250 CONNECTICUT AVENUE NW, SUITE 200, WASHINGTON, DC, 20036, US

(21) Appl. No.: 11/422,541

(22) Filed: Jun. 6, 2006

Related U.S. Application Data
(60) Provisional application No. 60/687,449, filed on Jun. 6, 2005.

(51) Int. Cl. A61K 39/02; A61K 31/739

(52) U.S. Cl. 424/234.1; 514/54

(57) ABSTRACT

The present invention relates to the ability of Francisella tularensis LVS lipopolysaccharide (LPS) to confer protection in a subject against a subsequent bacterial infection. Accordingly, the invention provides methods of treating and preventing bacterial infection in a subject by administering F. tularensis LPS to the subject. The invention also relates to methods of modulating the immune response in a subject comprising administering F. tularensis LPS to the subject.

### CROSS-REFERENCE TO RELATED APPLICATIONS

This application claims priority to U.S. Provisional Application Ser. No. 60/687,449, Jun. 6, 2005, which is incorporated by reference in its entirety.

### STATEMENT REGARDING FEDERALLY SPONSORED RESEARCH OR DEVELOPMENT

Part of the work performed during development of this invention utilized U.S. Government funds from NIH grant number AI057168. The U.S. Government has certain rights in this invention.

### BACKGROUND OF THE INVENTION

1. Field of the Invention

The present invention relates to the ability of Francisella tularensis lipopolysaccharide (LPS) to confer protection in a subject against a subsequent bacterial infection. Accordingly, the invention provides methods of treating and preventing bacterial infection in a subject by administering F. tularensis LPS to the subject. The invention also relates to methods of modulating the immune response in a subject comprising administering F. tularensis LPS to the subject.

2. Background of the Invention

Francisella tularensis (Ft) is a non-spore forming, encapsulated Gram negative coccobaccillus and the etiologic agent of the zoonotic disease tularemia. Since the 1950s, the incidence of tularemia in the United States has steadily declined, with only 1,368 cases of tularemia reported to CDC between 1990 and 2000; however, interest in tularemia has increased in recent years due its potential use as a bioweapon. Ft has been classified as by the U.S. Centers for Disease Control and Prevention as a Category A bioterrorism agent due to its ability to spread via the airborne route, its extremely low infectious dose, and its capacity to cause severe disease and death. The high infectivity of the fully virulent strain underlies the fact that the majority of research into the pathogenesis of Ft has utilized an attenuated “Live Vaccine Strain” (LVS). Ft LVS was developed in the former Soviet Union in the 1940's by repeatedly passaging the Type B strain of Ft on agar plates and then through mice. While Ft LVS is attenuated in humans, it is fully virulent in mice and causes a disease that resembles human tularemia.

For humans, natural tularemia infection can be acquired through direct contact with infected animals or contaminated hay, consumption of contaminated food or water, inhalation of contaminated air, or by the bite of an infected insect. The infectious dose required to cause human infection varies with route of entry. While respiratory infection can result from as few as 25 inhaled organisms, one must ingest upwards of 10⁸ bacteria in order to cause glandular infection. The severity of illness is also dependent upon the route of exposure as the pneumonic form of tularemia is most severe and has the highest mortality rate, greater than 30% when untreated. Similar to tularemia in humans, the outcome of Ft LVS exposure in mice is dependent on both the size of the inoculum as well as the route of inoculation. When mice are injected with Ft LVS intraperitoneal, the infection is nearly always fatal as the LD₅₀ is less than 10 organisms; however, the outcome is quite different when the bacteria are introduced subcutaneously or intradermal as estimates for the LD₅₀ range from 10⁵-10⁸ bacteria.

Infection with Ft also results in a pronounced inflammatory response. It has been suggested that this vigorous immune response is, in fact, responsible for most of the tissue damage associated with tularemia. Very little is known, however, about how Ft elicits this profound response, although recent evidence suggests that both innate and adaptive immune responses are involved. Specifically, SCID mice and IFN-γ knockout mice fail to survive intradermal infection with Ft LVS, suggesting an important role for the adaptive immune response in survival. More recently, MyD88 knockout mice were found to be similarly susceptible to intradermal infection, suggesting a role for Toll-like receptors (TLRs) in resistance to infection. Accordingly, the route of entry for Ft appears to have an impact on the outcome of infection in both human and mice.

What is needed in the art, therefore, are methods of protecting individuals from Ft infection or improving survival rate of individuals infected with Ft. What are also needed are methods of protecting the individual from the associated inflammatory cascade that inevitably follows Ft infection, as well as from inflammation, in general.

### SUMMARY OF THE INVENTION

The present invention relates to the ability of Francisella tularensis lipopolysaccharide (LPS) to confer protection in a subject against a subsequent bacterial infection. Accordingly, the invention provides methods of treating and preventing bacterial infection in a subject by administering F. tularensis LPS to the subject. In one aspect, the methods relate to treating and preventing bacterial infection in a subject by administering F. tularensis LPS to the subject, where the bacteria causing said bacterial infection is virulent and not F tularensis or the bacteria is F. tularensis. In another aspect, the LPS is purified from Live Vaccine Strain (LVS) of F. tularensis. In yet another aspect, the LPS is administered prior to exposure to the bacteria causing the infection.

The present invention also relates to methods of modulating the immune response in a subject comprising administering F. tularensis LPS to the subject. The present invention also relates to methods of enhancing the immunocompetence in a subject comprising administering F. tularensis LPS to the subject. The present invention also relates to methods of inhibiting the production of proinflammatory cytokines, such as, but not limited to IFN-γ, TNF-α, IL-4 and KC, in a subject comprising administering F. tularensis LPS to the subject.

The inventions also provide methods of treating a disease state in an individual, where the disease state is marked by inflammation, with the methods comprising administering lipopolysaccharide (LPS) from Francisella tularensis (F. tularensis) to said subject.

### BRIEF DESCRIPTION OF THE DRAWINGS

FIG. 1 depicts levels of F. tularensis 16S RNA in the liver, lung and spleen of mice inoculated with the bacteria, as a measure of bacterial burden after exposure. In general, mice exposed to F. tularensis LVS via intraperitoneal injection exhibited a higher bacterial burden.

FIG. 2 depicts levels of proinflammatory, chemokine and interferon mRNA in the liver, lungs and spleen of mice in response to F. tularensis LVS inoculation either via intraperitoneal or intradermal exposure. Specifically, FIGS. 2A and 2B show levels of proinflammatory mRNA in response to intraperitoneal or intradermal inoculation with F. tularensis respectively. FIGS. 2C and 2D show levels of chemokine mRNA in response to intraperitoneal or intradermal inoculation with F. tularensis respectively. FIGS. 2E and 2F show levels of interferon mRNA in response to intraperitoneal or intradermal inoculation with F. tularensis respectively.

FIG. 3 depicts the histology of livers 48 hours after exposure to F. tularensis in (a) a control mouse, (b) a mouse inoculated intradermally with F. tularensis LVS; and (c) a mouse inoculated intraperitoneally with F. tularensis LVS. Mice challenged with F. tularensis intraperitoneally had larger and more numerous granulomas that either control animals or animals challenged intradermally.

FIG. 4 depicts levels of an NF-κB-luciferase reporter signal in HEK293T cells transfected with various constructs, as an indication of the activation of the toll-like receptor2 and 4 (TLR2 and TLR4) F. tularensis LVS LPS does not appear to activate TLR2, and activates TLR4 only at the highest concentration tested (FIG. 4A), and it does not appear that F. tularensis LVS LPS blocks the activation of TLR4 by E. coli LPS (FIG. 4B). On the other hand F. tularensis LVS appears to activate TLR2 (FIG. 4C).

FIG. 5 depicts levels of F. tularensis 16S RNA in the liver of mice pretreated with F. tularensis LVS LPS prior to inoculation with F. tularensis LVS. Pretreatment of mice with F. tularensis drastically lowered bacterial burden of mice after exposure to LVS.

FIG. 6 depicts levels of proinflammatory gene expression, via mRNA levels, in the liver of mice pretreated with F. tularensis LVS LPS prior to inoculation with F. tularensis LVS. Pretreatment of mice with F. tularensis drastically reduced mRNA levels of all proinflammatory cytokines after exposure to LVS.

FIG. 7 depicts levels of proinflammatory gene expression, via mRNA levels (7A) and protein levels (7B), in vitro in macrophages from either wild-type mice or TLR2 knockout mice exposed to Ft LVS. TLR2 knockout mice had no response to Ft LVS, whereas wild-type mice increased levels of gene expression in response to Ft LVS.

FIG. 8 depicts mRNA levels of Toll-like Receptor 2 (TLR2) and TLR4 in vivo in wild-type mice. Levels of mRNA encoding TLR2 were upregulated in mice in response to Ft LVS, whereas levels of TLR4 remained unchanged.

FIG. 9 depicts the ability of viable Ft LVS to activate the NF-κB transcription factor. HEK293T cells, containing an NF-κB reporter (luciferase) construct, were transfected with TLR2 and then exposed to either viable, heat-treated, chloramphenicol (CAP)-treated or formalin-treated Ft LVS. After incubation, cells were lysed and luciferase activity was assayed and normalized against β-galactosidase activity. The data indicate that Ft LVS must be viable to activate the NF-κB transcription factor.

FIG. 10 depicts the ability of non-replicating Ft LVS mutant strain to activate the NF-κB transcription factor. HEK293T cells were transfected with TLR2 and exposed to either Ft LVS or an autotrophic mutant strain of Ft LPS that is deficient in the guaA gene (“ΔguaA mutant”) at the indicated MOI. Guanine was added to the indicated cultures prior to incubation with the Ft LVS or the ΔguaA mutant. After incubation, cells were lysed and luciferase activity was assayed and normalized against β-galactosidase activity. Since the ΔguaA mutant activates TLR2 to the same extent as wild-type Ft LVS, in the absence or presence of guanine, the data suggests that replicating Ft LVS is not necessary for activating the NF-κB transcription factor.

FIG. 11 depicts the survival rate of mice, pre-treated with Ft LVS LPS or saline, that were challenged with 1000 CFU of Ft LVS. The curve shows that administering Ft LVS LPS in doses as small as 0.1 ng/mouse intraperitoneally was able to protect the mice and extend survival rates.

FIG. 12 depicts the survival rate of either wild-type mice or mice that are B-cell deficient that were pretreated with Ft LVS LPS and subsequently challenged with 1000 CFU of Ft LVS. The curve shows that B cells appear to be important for conveying the protective effects of Ft LVS LPS in subjects.

FIG. 13 depicts the survival rate of wild-type mice treated with either Ft LVS LPS (10 ng or 100 ng) or Ft LVS Lipid A (10 ng or 100 ng) 2 days prior to challenge with Ft LVS. The curve shows that the Lipid A portion of the LPS alone extends survival rates of subjects.

FIG. 14 depicts the survival rate of wild-type mice treated with either saline or 100 ng of Ft LVS LPS 2 days prior to challenge with SHU S4. These data show that Ft LVS LPS offers animals partial protection against challenge with SHU S4.

### DETAILED DESCRIPTION OF THE INVENTION

The present invention relates to the ability of Francisella tularensis (Ft) LVS lipopolysaccharide (LPS) to confer protection in a subject against a subsequent bacterial infection. Accordingly, the invention provides methods of treating and preventing bacterial infection in a subject by administering F. tularensis LPS to the subject. As used herein, the term “treatment” is used to indicate a procedure which is designed ameliorate one or more causes, symptoms, or untoward effects of an abnormal condition in a subject. Likewise, the term “treat” is used to indicate performing a treatment. The treatment can, but need not, cure the subject, i.e., remove the cause(s), or remove entirely the symptom(s) and/or untoward effect(s) of the abnormal condition in the subject. Thus, a treatment may include treating a subject to attenuate symptoms of a bacterial infection, or it may include removing or decreasing the severity of the root cause of the abnormal condition in the subject. As used herein, the term “prevent,” as it relates to bacterial infection, indicates that a substance of the present invention is administered to a subject to prohibit one or more symptoms of a bacterial infection from detectably appearing or to attenuate the effects of one or more symptoms of bacterial infection. Of course, the term “prevent” also encompasses prohibiting entirely the bacterial infection or any of its associated symptoms, from detectably appearing. And “preventing” also includes providing a substance to a subject prior to any known exposure to a pathogen, such when a vaccination is administered. The phrase “preventing the progression,” as it relates to a bacterial infection, is used to mean a procedure designed to prohibit the detectable appearance of one or more additional symptoms of a bacterial infection in a patient already exhibiting one or more symptoms of a bacterial infection, and is also used to mean prohibiting the already-present symptoms of bacterial infection from worsening in the subject. As used herein, the term “subject” is used interchangeably with the term “patient,” and is used to mean an animal, in particular a mammal, and even more particularly a non-human or human primate.

Depending on the identity of the organism and its point of entry, the symptoms and indications of bacterial infection vary. Examples of organisms that cause bacterial infections, particularly in humans, includes but is not limited to Staphylococcus aureus, Staphylococcus epidermidis, Streptococcus pyogenes, Streptococcus pneumoniae, Listeria monocytogenes, Neisseria gonorrhoeae, Neisseria meningitides, Haemophilus influenzae, Pseudomonas aeruginosa, Bordetella pertussis, Escherichia coli, Vibrio cholerae, Salmonella ser. Typhimurium, Shigella boydii, Shigella dysenteriae, Shigella flexneri, Shigella sonnei. Clostridium tetani, Clostridium botulinum, Bacillus cereus, Bacillus anthracis, Corynebacterium diphtheriae, Mycobacterium tuberculosis, Borrelia burgdorferi, Rickettsia rickettsii, Rickettsia prowazekii and Francisella tularensis. In one embodiment of the present invention, the bacterial infection is an infection of Francisella tularensis.

Tularemia is caused by Ft infection, and there are a variety tularemia conditions and symptoms, based upon the pathogen's route of entry into the body. For example, ulceroglandular tularemia can form after colonization on the skin, glandular tularemia can form after colonization in the lymph nodes, oculoglandular tularemia can form after colonization in the eye, oropharyngeal tularemia can form after colonization in the mouth or throat, intestinal tularemia can form after colonization in the bowels, pneumonic can form after colonization in the lungs, and typhoidal tularemia can form as a system-wide disease. Typical symptoms of the various forms of tularemia, which are listed below include, but are not limited to:

Pneumonic symptoms include but are not limited to: fever chills fatigue headache body aches sore throat cough burning sensation or pain in chest. Ulceroglandular symptoms include but are not limited to: raised and red bumps, pus, open ulcer, swollen and tender lymph nodes, fever and chills. Glandular symptoms include but are not limited to: swollen and tender lymph nodes. Oculoglandular symptoms include but are not limited to: sensitivity to light, tearing, puffy eyelid, swelling and redness and sores in the eye, and swollen lymph nodes. Oropharyngeal symptoms include but are not limited to: irritated membranes in the mouth, sore throat, ulcers in the throat or on tonsils and swollen lymph nodes. Intestinal symptoms include but are not limited to: fever, abdominal pain, diarrhea and vomiting. Typhoidal symptoms include but are not limited to: fever, chills, headache, muscle aches, poor appetite, nausea, vomiting, diarrhea, abdominal pain and cough. Symptoms of progression from other types include but are not limited to: swollen lymph nodes, difficulty in breathing, bleeding, confusion, coma, organ failure, septic shock and death. A mild form of Pneumonia may also develop.

The symptoms of tularemia can start suddenly after contact with the bacterium, usually within 2 to 4 days. Initial symptoms may include headaches, chills, nausea, vomiting, high fever and exhaustion. Extreme weakness and profuse drenching sweats may also develop. Within two days, an inflamed blister appears at the infection site and rapidly fills with pus and may form a sore. Lymph nodes around the sore or sores enlarge and may produce pus, which may later drain. A rash may also appear at any time during the course of the disease.

The invention provides methods of treating and preventing bacterial infection in a subject by administering F. tularensis LPS (Ft LPS) to the subject. As used herein, the term “administer” and “administering” are used to mean introducing at least one compound into a subject. When administration is for the purpose of treatment, the substance is provided at the time of, or after the onset of, a symptom of bacterial infection, such as tularemia. The therapeutic administration of this substance serves to attenuate any symptom, or prevent additional symptoms from arising. When administration is for the purposes of preventing infection (“prophylactic administration”), the substance is provided in advance of any visible or detectable symptom. The prophylactic administration of the substance serves to attenuate subsequently arising symptoms or prevent symptoms from arising altogether. The route of administration of the compound includes, but is not limited to, topical, transdermal, intranasal, vaginal, rectal, oral, subcutaneous intravenous, intraarterial, intramuscular, intraosseous, intraperitoneal, epidural and intrathecal.

The methods of treating or preventing bacterial infection of the present invention also include coadministering Ft LPS with another agent. The term “coadminister” indicates that each of at least two compounds is administered during a time frame wherein the respective periods of biological activity or effects overlap. Thus the term includes sequential as well as coextensive administration of the compounds of the present invention. And similar to administering compounds, coadministration of more than one substance can be for therapeutic and/or prophylactic purposes. If more than one substance is coadministered, the routes of administration of the two or more substances need not be the same. Examples of agents that may be coadministered with Ft LPS include, but are not limited to, such known antibiotics as streptomycin, gentamicin, doxycycline and ciprofloxacin.

Lipopolysaccharide (LPS) is an integral component of gram-negative bacteria cell walls and is composed of Lipid A and a polysaccharide component. LPS elicits the inflammation response in animals, and is, in general, responsible for the toxicity associated with bacterial infection. In particular, the toxicity of LPS is associated with Lipid A, whereas the polysaccharide component of LPS is generally responsible for the immunogenicity of LPS. As used herein, the term lipopolysaccharide, or LPS, is used to mean an LPS molecule comprised of at least a lipid component and a polysaccharide component. The LPS may be present in/on a live cell such as, but not limited to, F. tularensis, F. tularensis live vaccine strain (LVS), or any another gram-negative bacteria, or the LPS may be present as part of a phospholipid layer or bilayer, such as, but not limited to, a synthetic cell membrane, a naturally occurring cell membrane, an endosome, a micelle and a liposome. Alternatively, the Ft LPS may be purified from any strain of Ft. General methods of isolating LPS from a bacterial cell membrane, such as those developed by Darveau and Hancock (J. Bacteriol. 155(2): 831-838 (1983)), Manthey, C. and Vogel S. N., J Endotoxin Res. 1:84-91(1994) and Westphal, O. and Jann, K., In Methods in Carbohydrate Chemistry. R. L. Whistler, ed. Academic Press, New York, pp. 83-91 (1965) which are hereby incorporated by reference, are well known in art. Thus Ft LPS includes LPS isolated from virulent strains as well as from a live vaccine strain (LVS) (“Ft LPS LVS”).

As used herein, the term “purified” as it relates to LPS indicates that the LPS has been subjected to fractionation or purification procedures, such as, but not limited to, those procedures disclosed above, to remove various other components, and which compositions substantially retains its expressed biological activity. Where the term “substantially purified” is used, this designation will refer to a composition in which the LPS forms the major non-solvent component of the composition. For example, “substantially purified LPS” indicates that more than about 50%, about 60%, about 70%, about 80%, about 90%, about 95% of the non-solvent component of a solution or composition is the LPS of the present invention.

Various methods for quantifying the degree of LPS will be known to those of skill in the art in light of the present disclosure. These include, for example, determining the specific activity of an active fraction, or assessing the amount of LPS within a fraction. One method for assessing the purity of a fraction is to calculate the specific activity of one fraction, to compare it to the specific activity of the initial extract, and to thus calculate the degree of purity, herein assessed by a “x-fold purification number” (i.e., 2-fold, 5-fold, 10-fold, 50-fold, 100-fold, 1000-fold, etc.). The actual units used to represent the amount of activity will, of course, be dependent upon the particular assay technique chosen to follow the purification and whether or not the expressed protein or peptide exhibits a detectable activity.

As used herein the term Ft LPS also encompasses detoxified Ft LPS, which, as the name implies, is Ft LPS with a reduced or removed toxicity. The invention is not limited by the methods of producing detoxified LPS, and includes methods such as, but not limited to, radiation treatment and alkaline treatment. Other methods of detoxifying LPS are been described in Bhattacharjee et al., J. Infec. Dis. 173:1157 (1996) and U.S. Pat. No. 4,929,604, which are hereby incorporated by reference. Still more methods of producing detoxified LPS include, but are not limited to, genetic alteration of bacterial stains which result in bacteria containing LPS whose lipid A component causes less biological toxicity (See Somerville et al., J. Clin. Invest. 97:359 (1996), which is hereby incorporated by reference).

As used herein, the term Ft LPS also encompasses derivative of Ft LPS. And a derivative of Ft LPS is a compound with a similar structure to Ft LPS that possess either similar or reduced immunogenic properties as Ft LPS or similar or reduced toxic properties as Ft LPS or both. Methods of generating LPS derivatives are well known in the art, and disclosed in Qureshi et al., J Biol. Chem. 260(9):5271-8 (1985) and Qureshi et al., J Biol. Chem. 257(19):11808-15 (1982), which are hereby incorporated by reference.

The methods of treating or preventing bacterial infection of the present invention also include administering Ft LPS as an adjuvant. Methods for administering an adjuvant for the purpose of modulating an immune response are well known in the art (See, Pulendran, B., et al. Trends in Immunology 22(1):41-47 (2001), which is hereby incorporated by reference) “Adjuvant” is used herein as it is in the art; namely, as an agent that is capable of modifying the biological effects of other agents. Accordingly, Ft LPS may be administered as an adjuvant that is capable of stimulating the immune system to enhance the immune response of another agent, such as, but not limited to, a vaccine. Thus, the invention provides methods of inhibiting bacterial infection by coadministering Ft LPS with another agent. In one embodiment, Ft LPS is coadministered with another agent that is capable eliciting a cell-mediated immune response in a subject. In a more specific embodiment, the Ft LPS is coadministered with another agent that is capable eliciting an antibody response in a subject. In an even more specific embodiment, disease-specific antigens are coadministered with Ft LPS. In another specific embodiment, Ft LPS is administered to enhance the immunogenicity of a vaccination. In still another embodiment, the Ft LPS is coadministered with another agent that is capable stimulating an innate immune response in a subject.

The protective effects of Ft LPS involve both an innate immune response and an adaptive immune response. Accordingly, the present invention provides methods of modulating an innate immune response in a subject comprising administering Ft LPS to a subject. The present invention also provides methods of modulating an adaptive immune response in a subject comprising administering Ft LPS to a subject. As used herein, “modulate” is used to mean a detectable alteration or change in the qualitative or quantitative characteristics of a biological process. The biological process that is modulated may be present in a cell, tissue, organ, organ system or subject. Furthermore, the degree and direction of modulation is irrelevant for the purposes of the present invention, provided the alteration is detectable. Thus, the invention also provides methods of enhancing the immunocompetence in subject comprising administering Ft LPS to a subject.

Ft LPS affects the expression of genes that are normally expressed during an inflammatory reaction such as but not limited to cytokines, cytochromes, clotting factors, tissue remodeling enzymes and cell signaling molecules such as inducible nitric oxide synthase (iNOS). Proinflammatory cytokines include, but are not limited to, interferon gamma (IFN-γ), tumor necrosis factor alpha (TNF-α), interleukin 4 (IL-4), IL-5, IL-6, IL-12, lymphotoxin, monocyte chemoattractant protein-1 (MCP-1), RANTES (regulated upon activation, normal T-cell expressed, and presumably secreted), migration inhibitory factor, immune protein 10 (IP-10) and cytokine-induced neutrophil chemoattractant (KC). Thus the present invention provides methods of modulating proinflammatory genes in a subject comprising administering Ft LPS to a subject. In one embodiment, the present invention provides methods of modulating the production of proinflammatory cytokines in a subject comprising administering F. tularensis LPS to the subject.

Many of the proinflammatory cytokines are produced by cells in response to toll-like receptor (TLR) activation. Toll-like receptors are a family of transmembrane receptors that are believed to be responsible for induction of the adaptive immune response, including but not limited to, cytokine production and co-stimulatory molecules. Moreover, TLR activation has been observed in response microbial challenge in both humans and mice. The involvement of TLR in the immune response, however, is far from settled. See Nemazee, D. et al., Nature, 441:E4 (2006) and Pasare C. and Medzhitov, R., Nature, 441: E4 (2006). The inventors have discovered that, with regard to F. tularensis, TLR2 is necessary for initiation of the proinflammatory cascade after Ft infection. Indeed, the absence of TLR2 activation in TLR2 knockout mice resulted in a drastic reduction of proinflammatory cytokines. Accordingly, the invention provides methods of modulating the production of Ft-induced proinflammatory cytokines comprising selectively blocking the activation of at least one member of the TLR family of receptors, for example, TLR2. TLR2 activation blocking can be accomplished in a variety of manners, including but not limited to, competitive inhibitors of the TLR binding site, allosteric inhibition of the TLR binding site and blockage of downstream effector molecules or adaptor proteins including but not limited to, NF-κB and MyD88.

Because Ft LPS has been shown to modulate proinflammatory cytokines, the methods of the present invention also relate to modulating inflammation in a subject. In one embodiment, the invention provides methods inhibiting the production of inflammatory mediators including but not limited to proinflammatory cytokines, prostaglandins, free radicals and nitric oxide. Thus the present invention also provides methods of treating diseases, such as but not limited to, autoimmune diseases that are marked by inflammation. Examples of diseases with indications of inflammation include, but are not limited to fibrosis, rheumatoid arthritis, lupus, ulcerative colitis, Crohn's disease, thyroiditis, and psoriasis. Thus, Ft LPS can be administered to diminish proinflammatory regulators associated with disease state inflammation.

Certain embodiments of the present invention relate to administering a pharmaceutically effective amount of a medicament substance that is capable of treating, preventing or preventing the progression of a bacterial infection. A medicament useful for the methods of treating, preventing or preventing the progression of a bacterial infection may be prepared by standard pharmaceutical techniques known in the art, depending upon the mode of administration and the particular disease to be treated. The medicament will usually be supplied as part of a sterile, pharmaceutical composition which will normally include a pharmaceutically acceptable carrier. This pharmaceutical composition may be in any suitable form, (depending upon the desired method of administering it to a subject). It may be provided in unit dosage form, will generally be provided in a sealed container and may be provided as part of a kit, which may include instructions for use and/or a plurality of unit dosage forms.

The pharmaceutical composition may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by admixing the active ingredient with the carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. For the preparation of solutions and syrups, excipients which may be used include for example water, polyols and sugars. For the preparation of suspensions oils (e.g. vegetable oils) may be used to provide oil-in-water or water in oil suspensions. In certain situations, delayed release preparations may be advantageous and compositions which can deliver, for example, AET or a derivative thereof in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols or oils. When formulated in an ointment, the active ingredient may be employed with either a paraffin or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets. The pharmaceutical compositions may contain preserving agents, solubilising agents, stabilising agents, wetting agents, emulsifiers, sweeteners, colourants, odourants, salts (substances of the present invention may themselves be provided in the form of a pharmaceutically acceptable salt), buffers, coating agents or antioxidants. They may also contain therapeutically active agents in addition to the substance of the present invention.

Dosages of the substance of the present invention can vary between wide limits, depending upon the disease or disorder to be treated, the age and condition of the individual to be treated, etc. and a physician will ultimately determine appropriate dosages to be used.

The invention also provides methods of identifying compounds which may be useful for treating, preventing or preventing the progression of a bacterial infection. The screening methods comprise (a) providing Ft LPS to a cell with immune or inflammatory activity and measuring such activity to establish a baseline activity, (b) providing a Ft LPS and a test agent to a second cell with the same activity measured in (a); and (c) comparing the level of measured activity in (a) with the level of measured activity in (b), where a difference in activity indicates that the test substance may be useful in treating, preventing or preventing the progression of a bacterial infection.

“Measuring immune or inflammatory activity” can be used to mean a quantitative measurement of cytokines or other immune effector molecules, for example, cytokines. The quantitative measurement includes, but is not limited to, mass, concentration, biological activity. Example of biological activities that may be used to quantify immune or inflammatory activity include, but are not limited to, chemotactic, cytotoxic, enzymatic or other biological activities, such as quantifiable activities that are used, for example, by the National Institute for Biological Standards and Control (NIBSC) in the United Kingdom for the quantification of interferon, cytokine and growth-factor activity. Other examples of methods of measuring immune or inflammatory activity include assays described herein, such as measuring mRNA species or protein levels that are known immune effector molecule and inflammatory molecules, such as but not limited to IFN-γ, TNF-α, IL-4 and KC. The differences in levels of immune or inflammatory activity may be equal to zero, indicating that there is no difference in immune or inflammatory activity. The difference may simply be, for example, a measured fluorescent value, radiometric value, densitometric value, mass value etc., without any additional measurements or manipulations. Alternatively, the difference may be expressed as a percentage or ratio of the measured value of the antigen to a measured value of another compound including, but not limited to, a standard. The difference may also be determined using in an algorithm, wherein the raw data is manipulated.

As used herein, the terms “substance” “agent” and “compound” may be used interchangeably. The types of substances that may be assayed for their use in treating, preventing or preventing the progression of a bacterial infection include, but are not limited to, carbohydrates such as monosaccharides, disaccharides, oligosaccharides and polysaccharides, proteins, peptides and amino acids, including, but not limited to, oligopeptides, polypeptides and mature proteins, nucleic acids, oligonucleotides, polynucleotides, lipids, fatty acids, lipoproteins, proteoglycans, glycoproteins, organic compounds, inorganic compounds, ions, and synthetic and natural polymers.

As used herein the term “cell” is used to indicate one or more cells, and can be used interchangeably with the term “cells”, “cell culture” and “cell line.” In addition, the cells used in the screening methods can be isolated cells in an in vitro cell culture, or the cells may be in situ, as part of an organ or tissue; or the cells may be in vivo as part of an organ or tissue in a live subject, such as, but not limited to a mouse, rat, dog or non-human primate. The cells used in the screening methods may also be manipulated, modified, fixed or even lysed at any time during the screening process, for example, subsequent to application of the test substance, but prior to measuring the activity to be assessed. Provided that assayed activity can be measured (e.g., gene expression), the cells can thus be prokaryotic or eukaryotic, including but not limited to bacterial cells, insect cells, mammalian cells, and even plant cells. A “test cell” is a cell to which a test substance has been applied; and “control cell” is a cell to which the same test substance has not been applied. The control cell may or may not be a genetically, phenotypically or metabolically normal cell, but the control cell should be the same cell type as the test cell.

Immune activity is used to mean any indication that at least a portion of an immune response is occurring or has occurred. Examples of immune activity include, but are not limited to the production of antibodies, generated of a cell-mediated immune response, increases or decreases in survival time of animals upon challenge to a pathogen and a differences in quality or quantity of a subject's symptoms associated with a pathogenic infestation to name a few. Inflammatory activity is used to mean any indication that at least a portion of inflammation reaction is occurring or has occurred. Examples of inflammatory activity include but are not limited to, expression of proinflammatory genes, generation of inflammatory molecules and differences in quality or quantity of a subject's symptoms associated with inflammation, for example in an experimental model of rheumatoid arthritis.

The examples provided herein are illustrative and not intended to limit the scope of the invention.

### EXAMPLES

Subjects and Reagents

Five to 6-wk-old C57BL/6J mice were purchased from the Jackson Laboratory (Bar Harbor, Me.). Mice were injected i.p. with purified Ft LVS LPS or inoculated i.p. or i.d. with Ft LVS bacteria. At the indicated times after inoculation, mice were sacrificed and livers, lungs, spleen, and blood were collected. Organs were snap frozen in an ethanol/dry ice bath and then stored at −80° C. for subsequent RNA extraction. Serum samples were collected and stored at −80° C. Serum cytokine concentrations were determined using ELISA assays or the Luminex® 100™ Total System (Luminex Corporation, Austin, Tex.) by the Cytokine Core Facility (Univ. of Maryland, Baltimore).

Peritoneal macrophages were isolated from mice 4 days after i.p. injection of sterile 3% thioglycollate as described previously in Salkowski, C. A., et al., J. Immunol. 163:1529-1536 (1999). Briefly, cells were washed in sterile 1×PBS and then resuspended in RPMI 1640 (Invitrogen, Carlsbad, Calif.) containing 2% FBS, 2 mM L-glutamine, 100 U/ml penicillin, and 100 μg/ml streptomycin. Macrophages were plated in 6 (4×106 cells/well) or 24 (1×106 cells/well) tissue culture plates (Corning Inc., Corning, N.Y.). After overnight incubation, cells were washed with 1× PBS to remove non-adherent cells. In experiments in which Ft LVS organisms were used in vitro, cells were cultured in antibiotic-free media both during and 24 hr prior to the experiment. Treatments were carried out in duplicate or triplicate. All animal experiments were conducted with Institutional Animal Care and Use Committee approval.

The synthetic lipoprotein S-[2,3-Bis(palmitoyloxy)-(2-RS)-propyl]-N-palmitoyl-(R)-Cys-(S)-Ser-Lys4-OH, trihydrochloride (Pam3Cys or P3C) was purchased from EMC Microcollections (Tuebingen, Germany). E. coli K235 LPS was prepared by the method of McIntire et al., Biochemistry 6:2363-2372 (1967). Ft LVS LPS was purified by List Biological Laboratories, Inc. (Campbell, Calif.). Briefly, the LPS was extracted from a wet cell pellet of Ft LVS using a modified Westphal/Jann protocol as described in Westphal, O. and K. Jann, In Methods in Carbohydrate Chemistry. R. L. Whistler, ed. Academic Press, New York, pp. 83-91 (1965). The aqueous fraction, which contained more than 75% of the LPS as determined by a KDO assay, was treated with RNAse, DNAse, and proteinase K. After treatment, the LPS mixture was centrifuged at 3,076×g to remove solids and diafiltered against 0.85% NaCl and distilled water using a 100 kDa AGT hollow fiber cartridge (Amersham Pharmacia/GE Healthcare; Sweden). Lyophilized LPS was then re-constituted in water and re-extracted using a modified deoxycholate-phenol extraction protocol as described in Manthey and Vogel Manthey, J Endotoxin Res. 1:84-91 (1994) and again subjected to treatment with DNase, RNAse, and proteinase K. Finally, the LPS was subjected to a chloroform:methanol (2:1) solvent extraction and dialyzed against water. Lyophilized LPS was resuspended in pyrogen-free saline at a concentration of 1 mg/ml for use in all assays.

Bacterial Infection Protocol

Frozen aliquots of Ft LVS are available from American Type Culture Collection, Manassas, Va. (ATCC 29684). F. tularensis LVS was cultured as described previously in Baker, et al., J. Clin. Microbiol. 22:212-215(1985) and Fortier, et al., Infect. Immun. 59:2922-2928 (1991), which are hereby incorporated by reference. Briefly, F. tularensis LVS was cultured on modified Mueller-Hinton (MH) agar plates or in modified MH broth (Difco Laboratories, Detroit, Mich.) supplemented with ferric pyrophosphate and IsoVitaleX (Becton Dickinson, Cockeysville, Md.). Aliquots of bacteria were frozen in broth alone and stored at −70° C. Bacterial concentrations were verified by plate count. For inoculations, bacteria were diluted in pyrogen-free saline and mice were injected either i.p. or i.d. with ˜5×10⁴ Ft LVS. Control animals were injected with saline only. Groups of 5 animals per treatment were sacrificed at the indicated times after inoculation. For the experiment in which mice were pretreated with Ft LVS LPS prior to infection, groups of 5 animals were injected with either saline or 100 ng Ft LVS LPS 2 or 7 days prior to i.p. challenge with 4×10⁴ Ft LVS. Forty-eight hours after Ft LVS challenge, all animals were sacrificed. All work involving Ft LVS was carried out under BSL2 conditions.

Real-Time PCR and Primers

Total RNA was isolated from both macrophages and organs using RNA Stat60. RT-PCR was performed in a Sequence Detector System (ABU Prism 7900 Sequence Detection System and software; Applied Biosystems, Foster City, Calif.). The protocol for RT-PCR was described previously in Cuesta, N., C., et al., J. Immunol. 170:5739-5747 (2003). Levels of mRNA are reported as “fold induction” over background levels detected in control samples unless otherwise noted. Primers were designed using the Primer Express™ Program (Applied Biosystems, Foster City, Calif.) in conjunction with GenBank sequences or were from the literature. All primers were synthesized at the Biopolymer Core Facility (Univ. of Maryland, Baltimore).

The following primer sets were used in our studies were as follows:

Ft 16srRNA sense
(5′-CAGCCACATTGGGACTGAGA-3′);
Ft 16srRNA antisense
(5′-CACACATGGCATTGCTGGAT-3′);
IFN-β sense
(5′-CACTTGAAGAGCTATTACTGGAGGG-3′);
IFN-β antisense
(5′-CTCGGACCACCATCCAGG-3′);
IFN-γ sense
(5′-CTGCCACGGCACAGTCATTG-3′);
IFN-γ antisense
(5′-TGCATCCTTTTTCGCCTTGC-3′);
IL-10 sense
(5′-ATTTGAATTCCCTGGGTGAGAAG-3′);
IL-10 antisense
(5′-CACAGGGGAGAAATCGATGACA-3′);
IL-1β sense
(5′-ACAGAATATCAACCAACAAGTGATATTCTC-3′);
IL-1β antisense
(5′-GATTCTTTCCTTTGAGGCCCA-3′);
IL-12p35 sense
(5′-GACGTCTTTGATGATGACCCTG-3′);
IL-12p35 antisense
(5′-TGTGATTCTGAAGTGCTGCGTT-3′);
IL-12p40 sense
(5′-TCTTTGTTCGAATCCAGCGC-3′);
IL-12p40 antisense
(5′-GGAACGCACCTTTCTGGTTACA-3′);
IL-4 sense
(5′-GCATTTTGAACGAGGTCACAGG-3′);
IL-4 antisense
(5′-TATGCGAAGCACCTTGGAAGC-3′);
IL-6 sense
(5′-TCAGGAAATTTGCCTATTGAAAATTT-3′);
IL-6 antisense
(5′-GCTTTGTCTTTCTTGTTATCTTTTAAGTTGT-3′);
iNOS sense
(5′-GTCTTTGACGCTCGGAACTGT-3′);
iNOS antisense
(5′-GATGGCCGACCTGATGTTG-3′);
IP-10 sense
(5′-CTTGGGATCCACACTCTCCAG-3′);
IP-10 antisense
(5′-TTTTTGGCTAAACGCTTTCATTAA-3′);
HPRT sense
(5′-GCTGACCTGCTGGATTACATTAA-3′);
HPRT antisense
(5′-TGATCATTACAGTAGCTCTTCAGTCTGA-3′);
KC sense
(5′-TGTCAGTGCCTGCAGACCAT-3′);
KC antisense
(5′-GCTATGACTTCGGTTTGGGTG-3′);
MCP-1 sense
(5′-TGGCTCAGCCAGATGCAG-3′);
MCP-1 antisense
(5′-GGTGATCCTCTTGTAGCTCTCCAG-3′);
RANTES sense
(5′-GAGTGACAAACACGACTGCAAGAT-3′);
RANTES antisense
(5′-CTGCTTTGCCTACCTCTCCCT-3′);
TNF-α sense
(5′-GACCCTCACACTCAGATCATCTTCT-3′);
TNF-α anti-sense
(5′-CCACTTGGTGGTTTGCTACGA-3′).

iNOS Staining of Liver Sections

Livers were removed and fixed in 10% buffered formalin (Sigma, St. Louis, Mo.), and embedded in paraffin. After deparaffinization, endogenous peroxidase activity was blocked with 3% hydrogen peroxide for 30 min at room temperature. For antigen unmasking, the slides were submerged in citrate buffer (pH 6) and heated for 10 min in a microwave oven. The sections were cooled in PBS and subsequently incubated with 3% non-fat dried milk for 30 min at room temperature to block non-specific binding. Slides were treated with rabbit polyclonal antibody against mouse iNOS (Santa Cruz Biotechnology, Inc, Santa Cruz, Calif.) at a 1:750 dilution at 4° C. overnight. The next day, sections were incubated with biotinylated goat anti-rabbit IgG (Vector Labs, Burlingame, Calif.; 1:200) and with avidin-biotin peroxidase complexes (Vector Labs; 1:100) for 30 min at room temperature. Peroxidase activity was visualized with DAB (Vector Labs) and nuclear counterstaining was performed with Harris hematoxylin. The slides were dehydrated and permanently mounted with Permount (Fisher Scientific, Fair Lawn, N.J.).

Plasmid Constructs and Reporter Assay

HEK293T cells were cultured in DMEM (BioWhittaker, Walkersville, Md.) supplemented with 10% FBS, 2 mM L-glutamine, 100 U/ml penicillin, and 100 μg/ml streptomycin. All cells were maintained in a 37° C. humidified atmosphere with 5% CO₂. HEK293T cells were transfected with plasmids encoding various TLRs, co-receptor, and reporter constructs as described previously Medvedev, A. E. and S. N. Vogel, J. Endotoxin. Res. 9:60-64 (2003). The constructs used include: pFLAG_TLR2, pcDNA3-TLR4, pcDNA3-huCD14 (CD14),pELAM_Luc, and pCMV1-β-gal, pEFBOS-HA-huMD-2 (MD-2), and pcDNA3 (empty vector; Invitrogen, Carlsbad, Calif.). All plasmid DNA was isolated using Endo-Free™ plasmid prep kits (Qiagen Inc., Valencia, Calif.). Briefly, 2×105 HEK293T cells were seeded into each well of 12-well costar plates (Corning Inc, Corning, N.Y.). After an overnight incubation, cells in each well were transfected for 3 hr with the various plasmids and pcDNA3 such that the total amount of DNA was 1.5 μg/well. Transfection was carried out with SuperFect transfection reagent (Qiagen Inc., Valencia, Calif.). After incubation overnight for recovery, cells were stimulated with LPS (Ft LVS or E. coli), Pam3Cys, or Ft LVS bacteria for either 5 or 24 hr. Cells were lysed in 1× reporter assay lysis buffer (Promega Corporation, Madison, Wis.), and β-gal (Galacto-Light system, Tropix, Bedford, Mass.) and luciferase (Luciferase assay system, Promega) activities were analyzed using a Berthold LB 9507 Luminometer (Berthhold Technologies, Bad Wildbad, Germany). “Relative luciferase activity” was calculated by normalizing each sample's luciferase activity with the constitutive β-galactosidase activity measured within the same sample.

Results—F. tularensis (LVS) Infection Increases Proinflammatory Cytokine Gene Expression in vivo

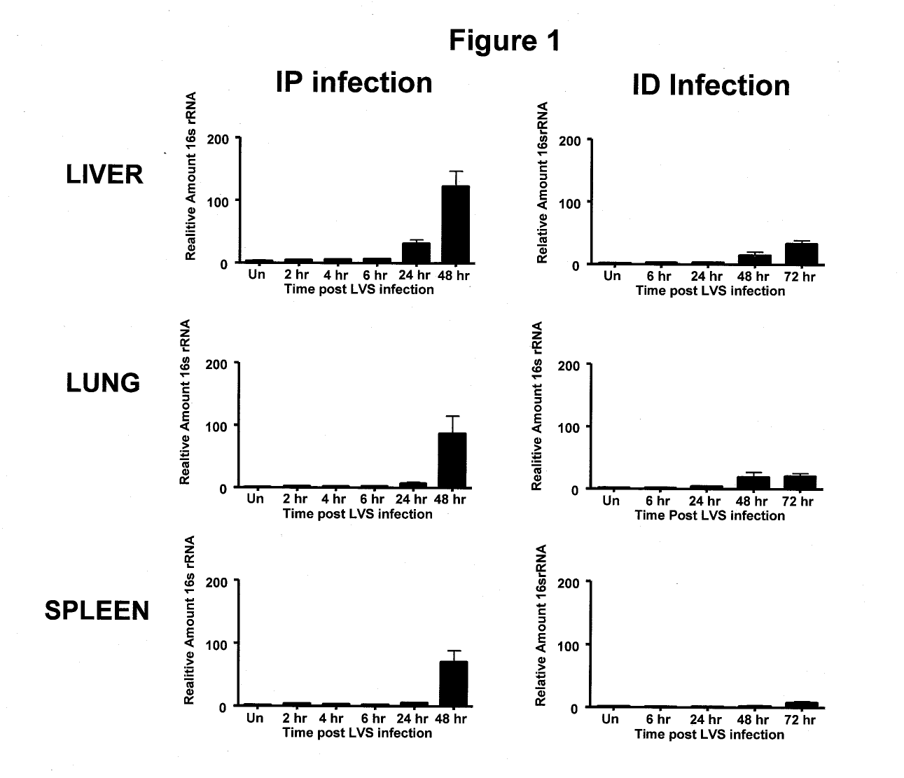

Initially, we compared the expression of Ft-specific 16S ribosomal RNA as an indirect measure of bacterial burden in the total RNA derived from the organs of mice inoculated i.p. or i.d. with 5×10⁴ CFU/mouse. FIG. 1 illustrates that the level of Ft LVS-specific mRNA was greatest in mice injected i.p., and that the liver contained the greatest amount of specific transcripts regardless of route of injection. Even at 72 hr post-infection, the amount of 16S rRNA was considerably lower in mice infected i.d. than in the 48 hr samples from mice infected i.p. Regardless of the route of inoculation, the liver expressed more 16S rRNA than the lung and spleen.

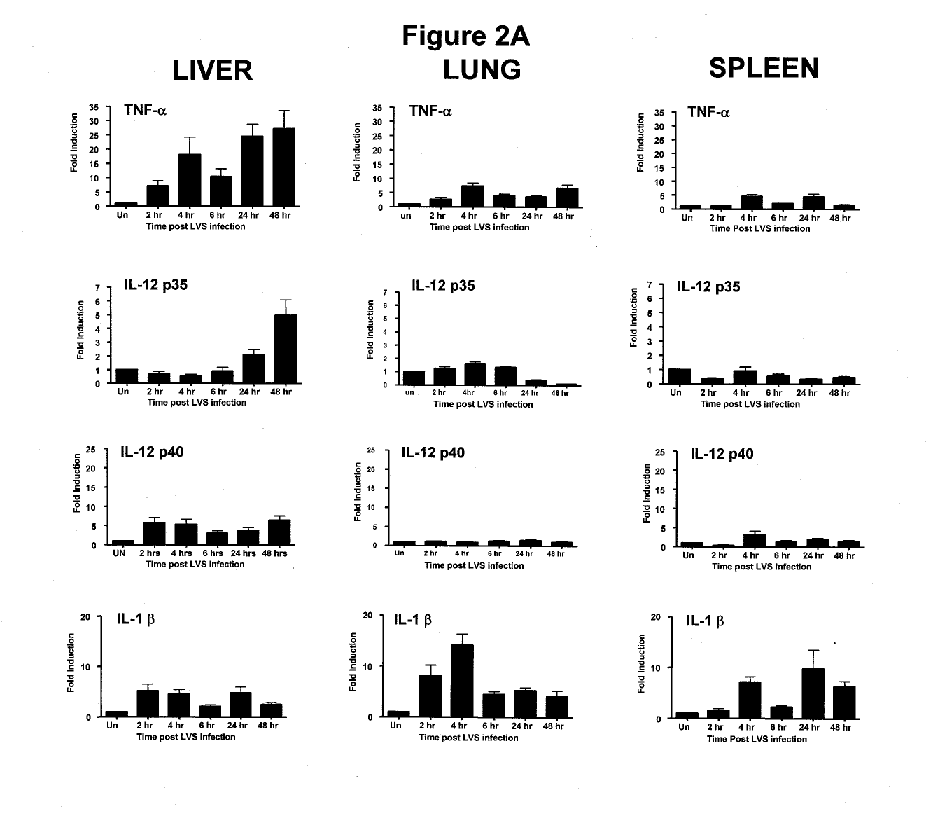

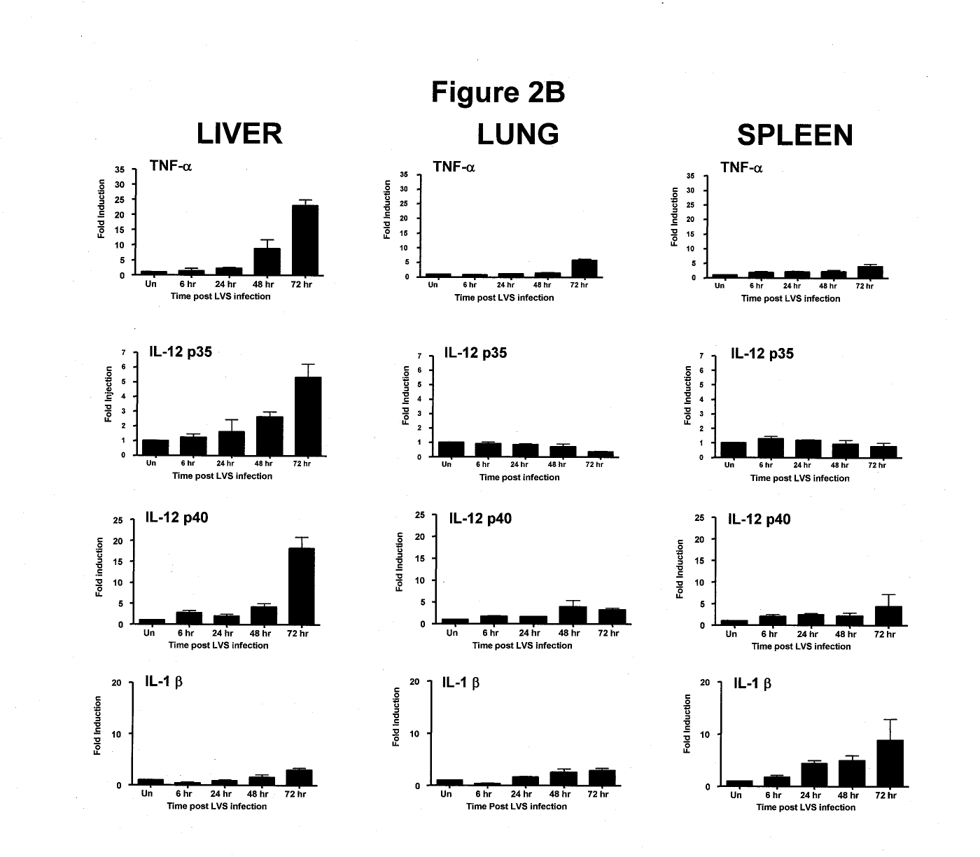

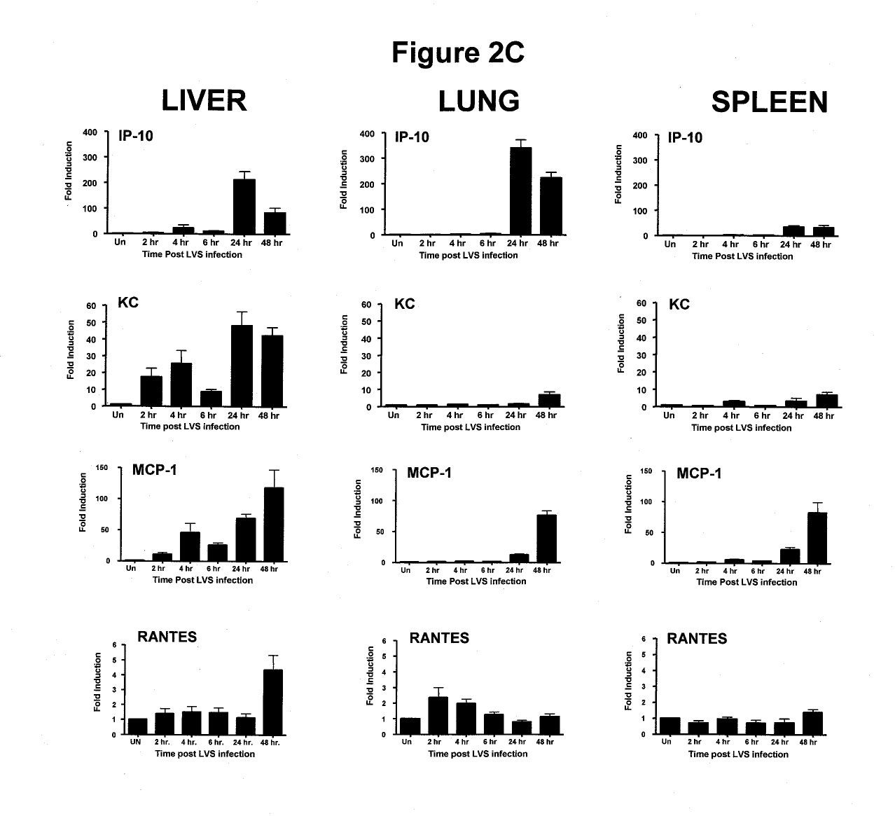

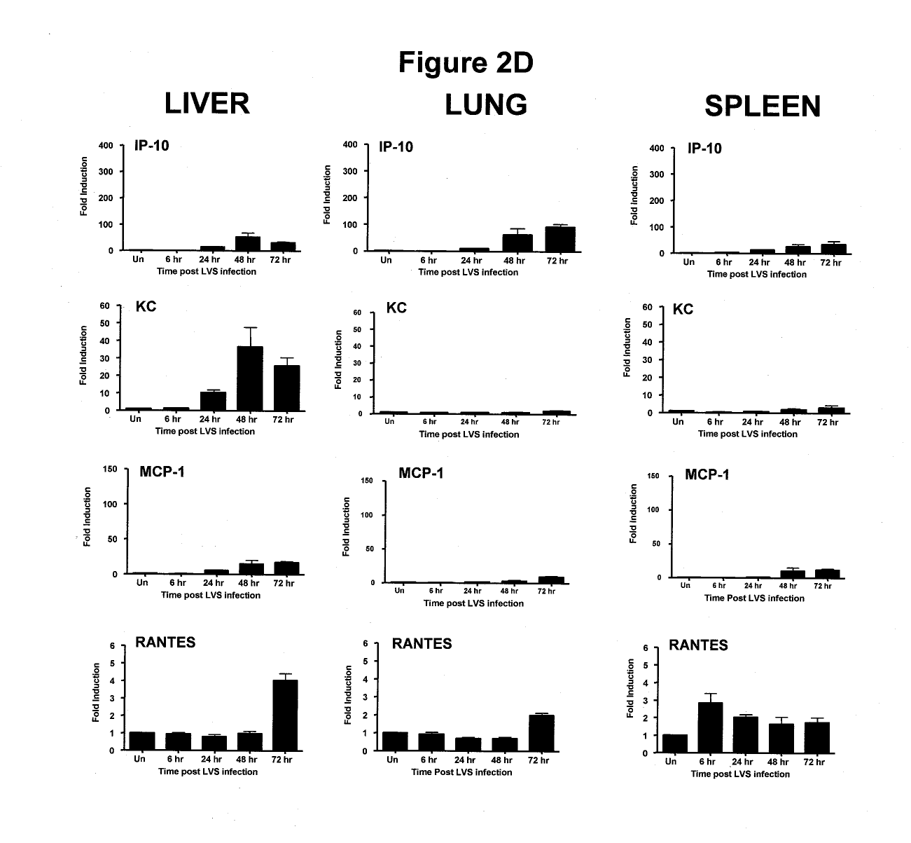

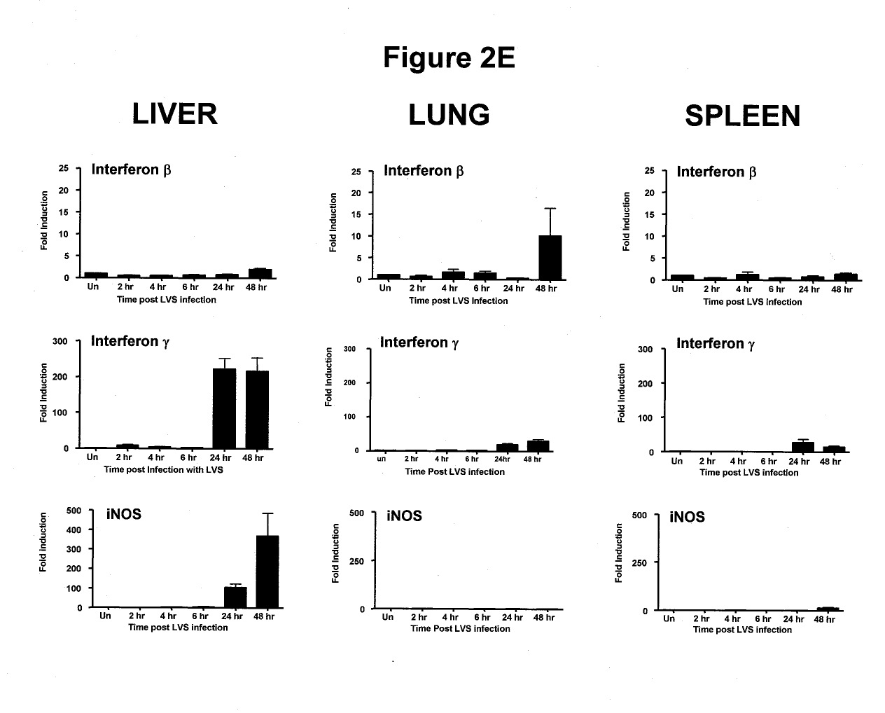

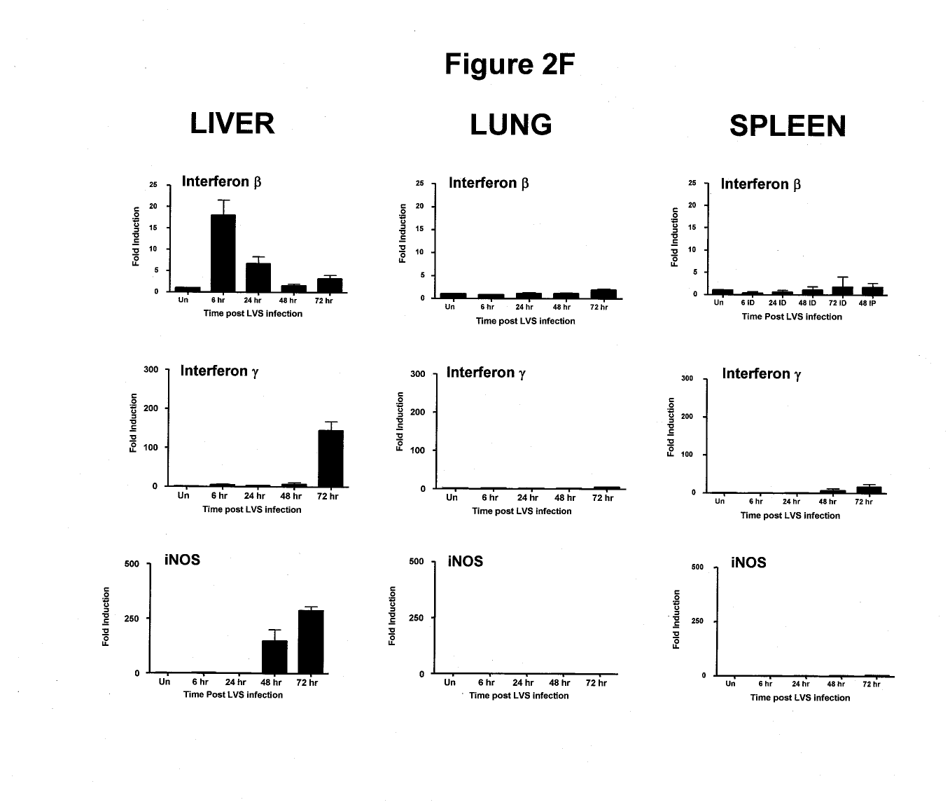

Intraperitoneal inoculation of Ft consistently induced greater levels of inflammatory gene expression (FIGS. 2A-F; summarized in Table I). The greatest disparity in differences in gene expression due to route of inoculation was seen with the gene that encodes the chemokine, MCP-1, which displayed a greater than 100-fold induction in liver and nearly a 100-fold induction in both spleen and lung at 48 hours post inoculation. (FIG. 2C vs. 2D). In contrast, the average increase in MCP-1 mRNA for mice inoculated i.d. was 14-, 3-, and 10-fold for liver, lung, and spleen, respectively at the same time points.

In both sets of animals, pro-inflammatory genes, save for IP-10, were upregulated in a time-dependent fashion, with the greatest increase seen at later time points, which is consistent with the increase in bacterial burden shown in FIG. 1.

IFN-γ, iNOS, KC, and TNF-α mRNA species were most strongly unregulated in the livers of mice inoculated by either route, while there was far less expression of these specific genes in lungs and spleens (Table I).

TABLE I
Induction of various genes in response
to i.p. or i.d. inoculation of mice with 5 × 10⁴.

| | | Liver | Liver | Lung | Lung | Spleen | Spleen |
|---|---|---|---|---|---|---|---|
| | Gene | (i.p.) | (i.d.) | (i.p.) | (i.d.) | (i.p.) | (i.d.) |
| Pro- | TNF-α | ++ | ++ | + | + | + | −/+ |
| Inflammatory | IL-12 | + | + | − | − | − | − |
| | p35 | | | | | | |
| | IL-12 | + | ++ | − | −/+ | −/+ | −/+ |
| | p40 | | | | | | |
| | IL-1β | + | −/+ | ++ | −/+ | ++ | + |
| Interferons | IFN-β | − | ++ | + | − | − | − |
| | IFN-γ | ++++ | ++++ | ++ | −/+ | ++ | ++ |
| Chemokines | IP-10 | ++++ | ++ | ++++ | +++ | ++ | ++ |
| | KC | ++ | ++ | + | −/+ | + | −/+ |
| | MCP-1 | ++++ | ++ | +++ | + | +++ | + |
| | Rantes | + | −/+ | −/+ | −/+ | − | −/+ |
| Anti- | IL-4 | −/+ | − | −/+ | − | − | − |
| inflammatory | IL-10 | −/+ | −/+ | ++ | ++ | −/+ | + |
| Others | iNOS | ++++ | ++++ | − | − | + | −/+ |

Symbol Legend

| | | | | | | |
|---|---|---|---|---|---|---|
| Average | 1 | >1 to ≦5 | >5 to ≦10 | >10 to ≦50 | >50 to ≦100 | ≦100 |
| Fold | | | | | | |
| Induc- | | | | | | |
| tion | | | | | | |
| Symbol | − | −/+ | + | ++ | +++ | ++++ |

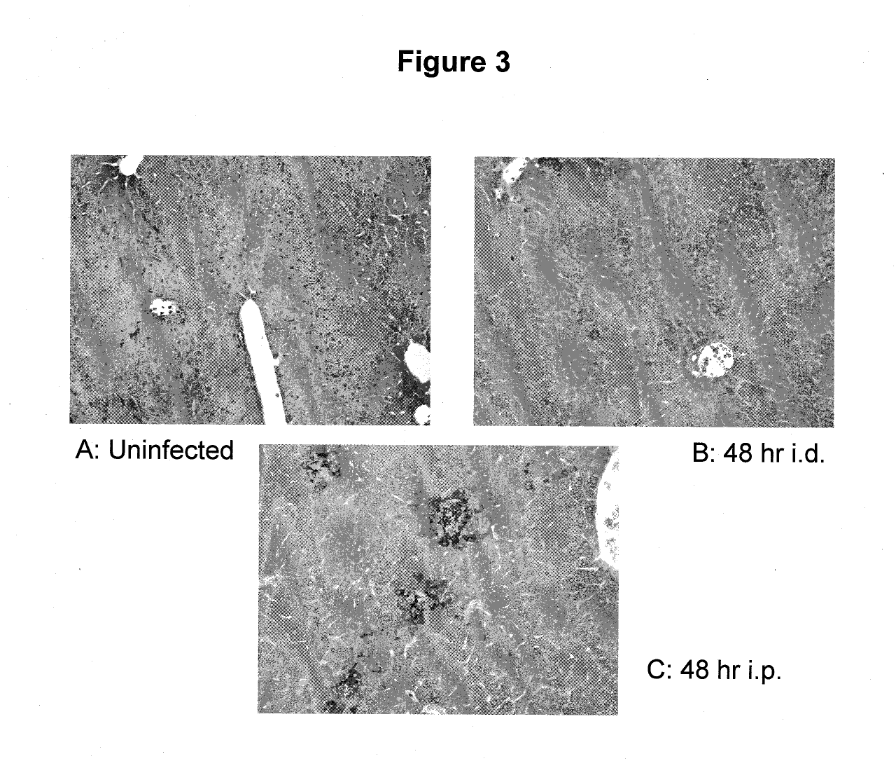

At 48 hours post inoculation, mice inoculated i.p. exhibited larger and more numerous granulomas than either control animals (saline injection) or animals inoculated i.d. (FIG. 3). In addition, only mice infected i.p. developed liver granulomas that expressed iNOS intracellularly.

We also measured several cytokines in the sera of animals infected i.p. to confirm production of cytokines at the level of protein. Consistent with the results measured at the level of mRNA, mice infected i.p. produced significant levels of circulating TNF-α (75.0±36.1 pg/ml) and Rantes (777.6±32.5 pg/ml) by 48 hr post infection, compared to the concentrations for control animals that was below the limit of assay detection (7.8 pg/ml) for TNF-αand <80 pg/ml for Rantes.

Results—F. tularensis LVS Signals via TLR2 and F. tularensis LVS LPS Does Not

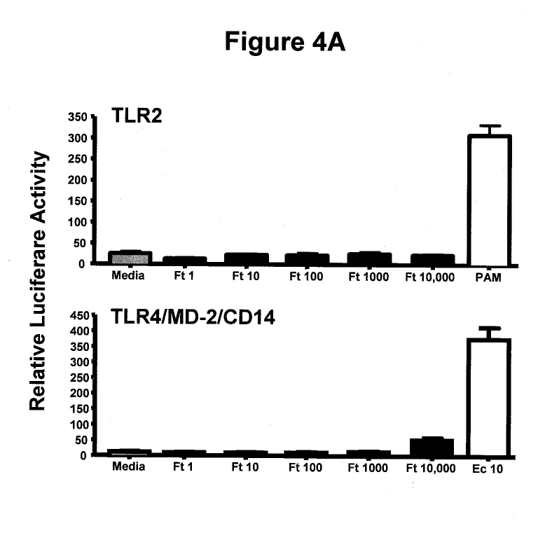

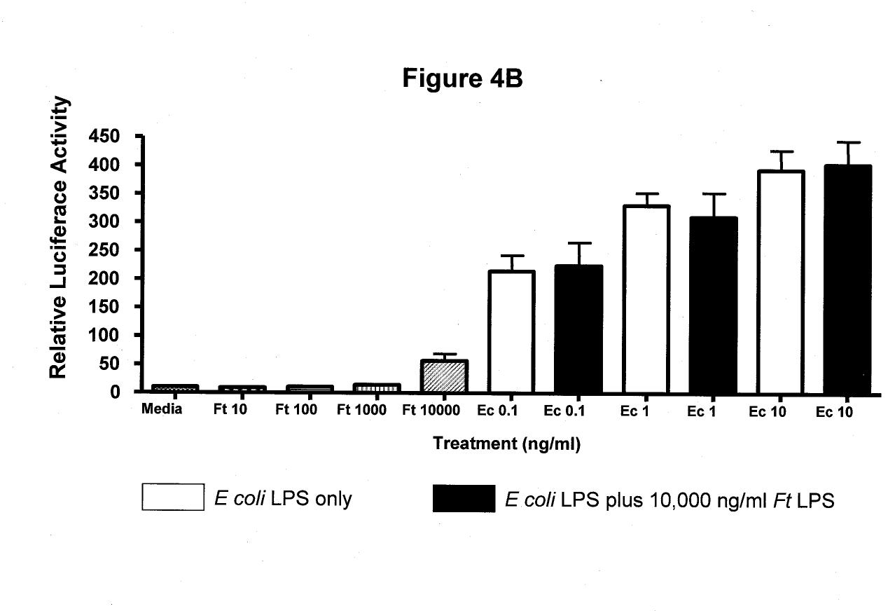

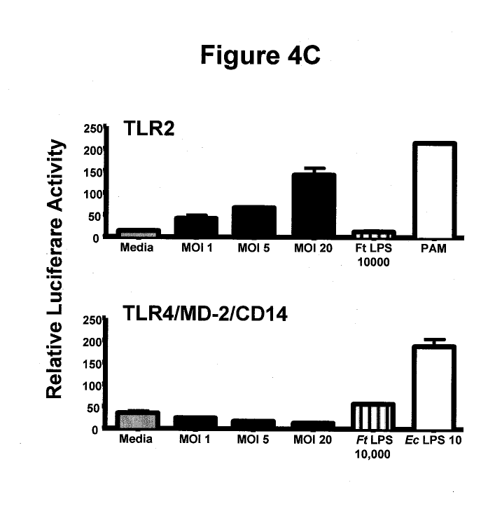

HEK293T cells were transfected with plasmids encoding either human TLR2 or TLR4/MD-2/CD14 to reconstitute TLR2 and TLR4 signaling complexes. Ft LVS LPS was unable to activate HEK293T cells transiently tranfected with TLR2, but activated cells tranfected with TLR4/MD-2/CD14 only at the highest concentration of Ft LVS LPS tested, i.e., 10,000 ng/ml (FIG. 4A). This level of Ft LPS activation, however, is minimal compared to E. coli LPS, which induced a 10-fold greater activation of the NF-κB luciferase reporter at a concentration that was 1000-fold less. In addition, no signaling through TLR3, TLR5, TLR7, or TLR8 was observed when tested in the same system (data not shown).

We next used the HEK293T transient transfection system to determine if live Ft LVS bacteria, rather than the purified Ft LVS LPS, signaled though either TLR2 or TLR4. In contrast to the results seen with Ft LVS LPS alone, live Ft LVS were able to signal through TLR2 in a dose-dependent manner (FIG. 4C). While Ft LVS activation through the TLR2 receptor was not as great as activation seen in the positive control (1 μg/ml Pam3Cys), Ft LVS activation was nonetheless significantly greater than the response achieved by treatment of cells with media alone (p<0.0001) all three MOI tested.

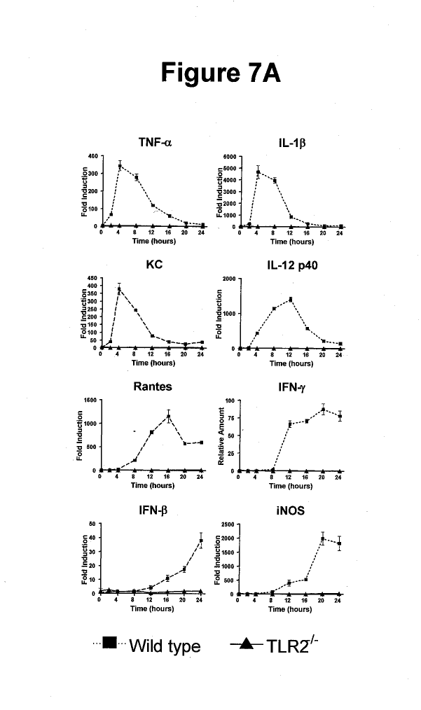

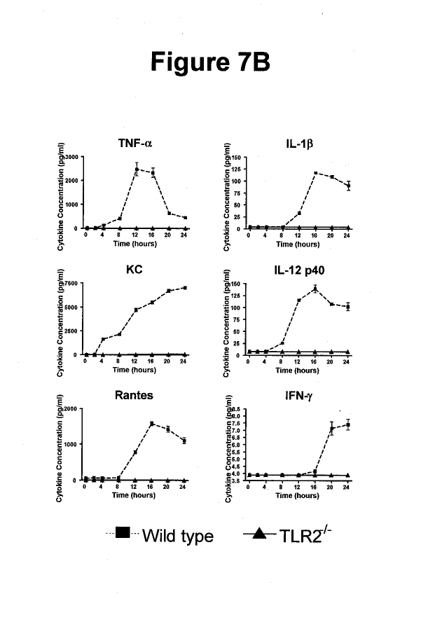

Next, we sought to determine the extent to which macrophage cytokine gene expression was also dependent on TLR2. Thioglycollate-elicited macrophages derived from either wild-type or TLR2^{-/-} knockout mice were stimulated with live Ft LVS (MOI 5) for 0 to 24 hr and proinflammatory gene expression and cytokine production measured. As can be seen in FIG. 7A, real time PCR revealed three general patterns of cytokine mRNA expression in macrophages derived from wild type animals: expression that peaked prior to 8 hours (IL-1β, TNF-α, KC), between 8 and 12 hours (IL-12p35 and IL-12p40), and after 12 hours (IFN-β3, IFN-γ, iNOS, IP-10, and RANTES). In contrast, macrophages derived from TLR2^{-/-} mice displayed baseline mRNA expression of the same genes after exposure to Ft LVS throughout the time course (FIG. 7).

Protein concentrations of several cytokines in the macrophage culture supernatants were analyzed via ELISA. While the supernatants of wild-type macrophages contained significantly elevated concentrations of IFN-γ, IL-1β, IL-12p40, KC, RANTES, and TNF-α, there was no detectable IFN-γ, IL-1β, IL-12p40, and TNF-α and very little KC or RANTES in supernatants collected from TLR2^{-/-} macrophages (FIG. 7B). This again supports the conclusion that Ft LVS induced macrophage production of cytokines is overwhelmingly TLR2-dependent.

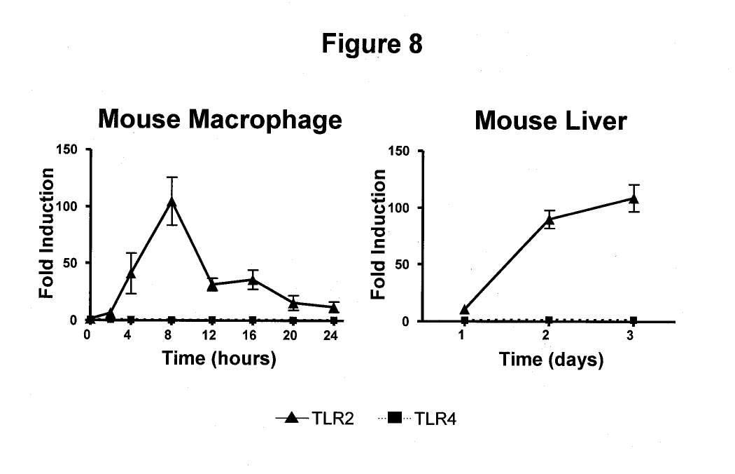

Interestingly, exposure to Ft LVS increased expression of TLR2 mRNA 100-fold in wild-type macrophages, while expression of TLR4 mRNA actually decreased relative to the media only control (FIG. 8). To determine if this was also the case in vivo, mice were injected intraperitoneally with ˜40,000 CFU, and the animals sacrificed one, two, or three days later. RNA was extracted from the livers and TLR2 and TLR4 mRNA expression was analyzed via real time PCR. As was observed in vitro, TLR2 mRNA expression increased in a time-dependent fashion, while TLR4 expression remained at basal levels (FIG. 8). These data indicate that Ft LVS is able to increase the expression of the gene that encodes its own receptor.

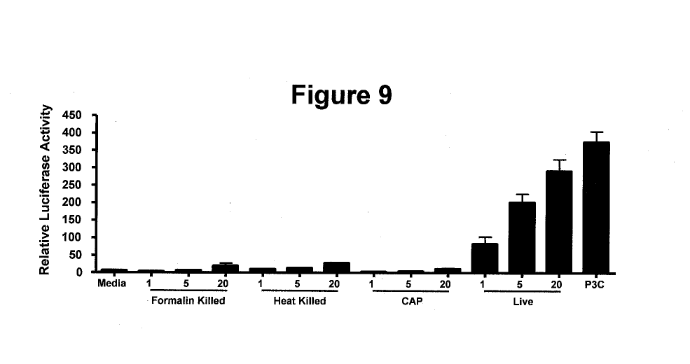

We next sought to test the requirement for bacterial viability for TLR2-dependent signaling induced by Ft LVS. When Ft LVS were killed with heat or formalin, the activation of the NF-κB luciferase reporter in HEK293T cells transiently transfected with TLR2 was significantly reduced (FIG. 9), indicating that only viable bacteria are able to activate NF-κB through TLR2.

We next tested the requirement for de novo protein synthesis. Chlorampheniol (CAP; 20 μg/ml), an antibiotic that inhibits bacterial protein synthesis, significantly reduced Ft LVS mediated NF-κB activation (FIG. 9), but had no impact on Pam3Cys-mediated activation (data not shown). This indicated that the CAP solution was specifically interfering with Ft LVS-mediated activation of NF-κB and was not generally inhibiting activation. Although CAP was solubilized in 100% ethanol, the inhibition was due to the CAP, as an equivalent volume of ethanol alone had no impact of Ft LVS activation of the NF-κB (data not shown). Taken together, these data indicate the Ft LVS must be viable and able to synthesize protein in order to activate NF-κB via TLR2.

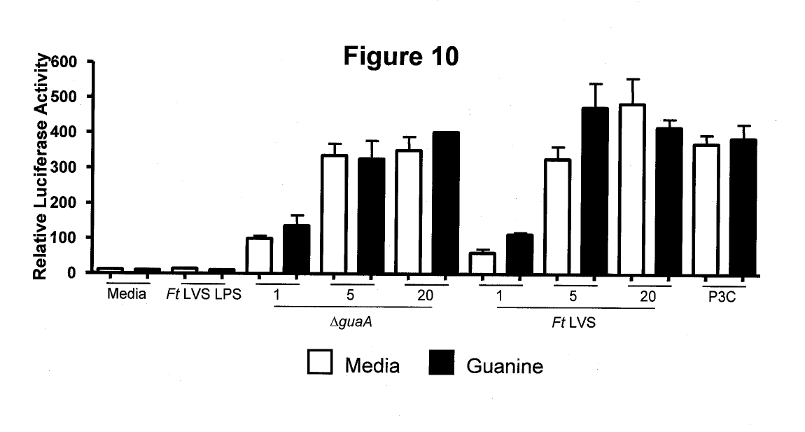

Recent efforts to develop a vaccine for F. tularensis have led to creation of a GuaA mutant strain of Ft LVS. The guaA gene encodes a critical enzyme in the guanine nucleotide biosynthetic pathway. The mutant strain was created by deleting the entire guaA gene and replacing it with a kanamycin resistance cassette, this strain is believed to be both autotrophic for guanine and attenuated for virulence in mice. Without the addition of exogenous guanine, ΔguaA is unable to grow in media or replicate in J774 macrophages. Therefore, we utilized this auxotrophic mutant to determine the requirement for bacterial replication for activation of NF-κB through TLR2. Regardless of the presence of guanine, the ΔGuaA mutant strain was able to activate NF-κB through TLR2 (FIG. 10), demonstrating that neither extracellular nor intracellular replication of Ft LVS is required for signaling through TLR2.

Results—F. tularensis LVS upregulates Proinflammatory Cytokines in Macrophages

Peritoneal macrophages were exposed to live Ft LVS for 24 hr at MOI of 1, 5, and 20. Analysis by real-time PCR showed that proinflammatory genes were strongly upregulated after exposure to Ft LVS, even at an MOI of 1 (Table II). These results sharply contrast with that seen in response to Ft LVS LPS, which had no effect on proinflammatory gene expression in macrophages. Supernatants were analyzed by ELISA and the concentrations of IFN-γ, IL-1β, IFN-γ, IL-12 p40, IL-12 p70, TNF-α and Rantes were consistent with the real-time PCR data.

TABLE II
Murine macrophages exposed to live Ft LVS upregulate expression of various
pro-inflammatory genes

| | | Ft LVS | Ft LVS | Ft LVS | Ft LPS | Ec LPS |
|---|---|---|---|---|---|---|
| | Gene | MOI 1 | MOI 5 | MOI 20 | 10,000 ng/ml | 10 ng/ml |
| Pro-Inflammatory | TNF-α | 30 | 37 | 35 | 1 | 21 |
| | IL-12 p35 | 175 | 763 | 3202 | 1 | 1754 |
| | IL-12 p40 | 97 | 123 | 293 | 7 | 1862 |
| | IL-1β | 165 | 378 | 2394 | 4 | 891 |
| | IL-6 | 6 | 45 | 634 | 1 | 41 |
| Interferons | IFN-β | 3 | 5 | 28 | 1 | 4 |
| | IFN-γ | 14* | 42* | 72* | 0.2* | 5* |
| Chemokines | IP-10 | 665 | 644 | 558 | 12 | 2223 |
| | KC | 13 | 45 | 249 | 1 | 6 |
| | MCP-1 | 6 | 24 | 25 | 1 | 123 |
| | Rantes | 776 | 2426 | 3349 | 266 | 23951 |
| Anti-Inflammatory | IL-4 | 0.9 | 0.3 | 0.12 | 1 | 0.3 |
| | IL-10 | 32 | 9 | 11 | 4 | 8 |
| Others | iNOS | 278 | 1700 | 6200 | 4 | 4175 |

*Relative amount of message reported based on extrapolation from a standard curve. All other genes are expressed as “fold induction” above levels present in medium-treated cells.

Results—Pretreatment of Subjects with F. tularensis LVS LPS Confers Protective Immunity Against Subsequent F. tularensis Infection

Groups of 5 mice were injected i.p. with either saline or 100 ng Ft LVS LPS 2 or 7 days prior to i.p. challenge with 4×10⁴ CFU Ft LVS (Table III).

TABLE III
Protocol for Ft LVS LPS pretreatment experiment.

| Day-7 | Day-2 | Day 0 | Day 2 |
|---|---|---|---|
| Ft LVS LPS | | Saline | Sacrifice |
| Ft LVS LPS | | Ft LVS | Sacrifice |
| Saline | | Saline | Sacrifice |
| Saline | | Ft LVS | Sacrifice |
| Saline | Ft LVS LPS | Saline | Sacrifice |
| Saline | Ft LVS LPS | Ft LVS | Sacrifice |

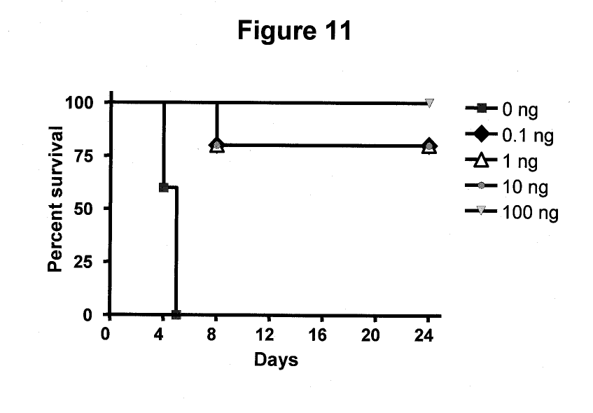

FIG. 11 shows the survival curve of mice injected with protective levels of Ft LVS LPS 2 days prior to an intraperitoneal challenge with 1000 CFU of Ft LVS. As little as 0.1 ng of Ft LVS LPS was able to protect and extend the survival rate of mice challenged with an otherwise lethal dose of Ft LVS. Administering Ft LVS LPS (0.1 ng-10 ng) nearly doubled the mean time of death from about 4.6 days to about 8 days.

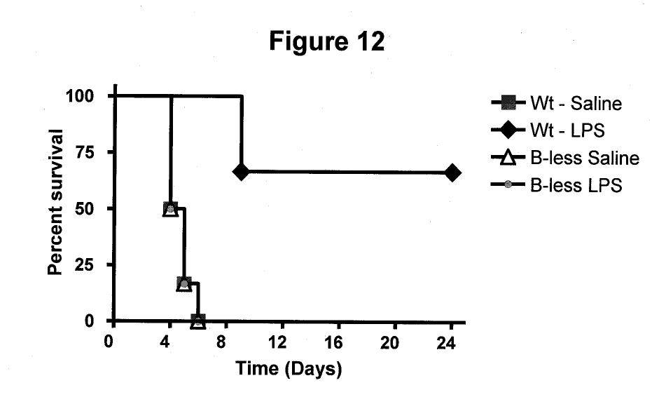

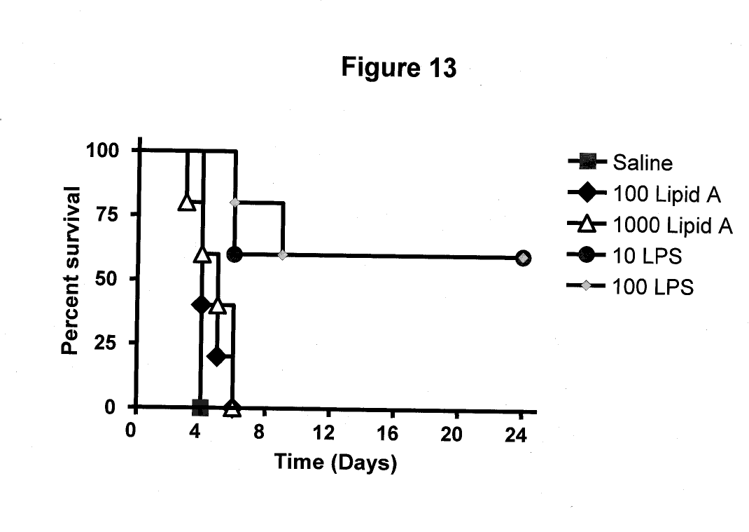

The protection afforded by pre-treatment of animals with Ft LVS LPS appears to be B-cell dependent. Indeed, Groups of 6 wild type (C57BL/6J Wt) or Igh-6tmlCgn targeted mutant animals (Jackson Laboratories, Bar Harbor, Me.), which are unable to produce mature B cells (B-less) were injected intraperitoneally with 100 ng Ft LVS LPS or saline two days prior to a intraperitoneal challenge with Ft LVS (1000 CFU). While 100 ng Ft LVS LPS afforded protection to the wild type animals, B-less mice died in the same time frame as wild type mice injected with only saline (FIG. 12). There was a statistically significant difference in the survival curves (P=0.0292). These results show that mature B cells are required for Ft LVS LPS mediated protection against an otherwise lethal challenge with Ft LVS.

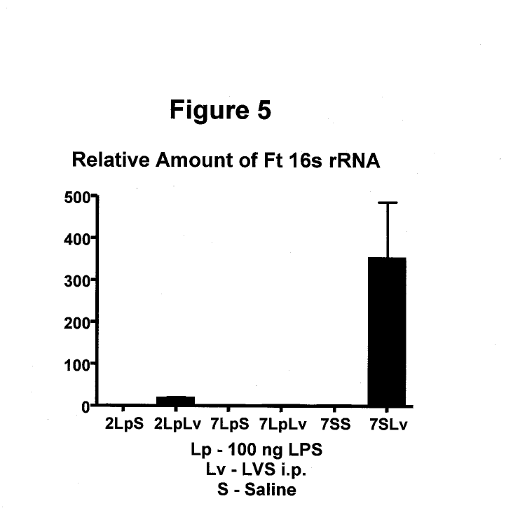

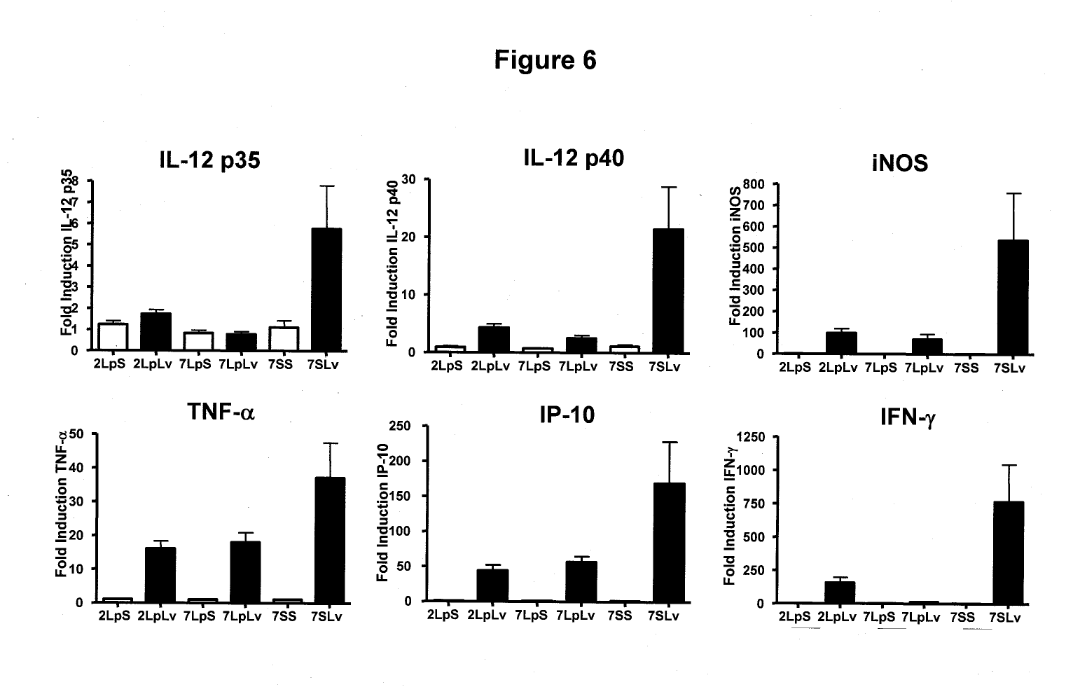

Forty-eight hr after challenge, all animals were sacrificed. As livers had been the primary site of pro-inflammatory gene expression in response to i.p. infection (FIG. 2 and Table I), they were analyzed for bacterial burden and gene expression. FIG. 5 illustrates that pretreatment with Ft LVS LPS strongly blunts the bacterial burden in livers of mice subsequently infected with Ft LVS i.p. Specifically, Ft-specific 16S mRNA was undetectable in mice pretreated with LPS 7 days prior to infection, and a 2-day pretreatment significantly decreased the amount of detectable 16S ribosomal RNA. In parallel with the reduced bacterial burden, the induction of all cytokines in liver was markedly inhibited (FIG. 6). In all cases, pretreatment of mice with Ft LVS LPS led to significantly lower levels of gene expression upon subsequent infection, compared to control mice (Table IV).

TABLE IV
Pretreatment of mice with 100 ng Ft LVS LPS i.p. blunts upregulation of pro-
inflammatory genes in response to Ft LVS infection.

| | | Pretreatment | | | | | |
|---|---|---|---|---|---|---|---|
| | | −2 | −2 | | −7 | −7 | |
| | | LPS | LPS | −7 LPS | LPS | Saline | −7 Saline |
| | | Day O | | | | | |
| | | Saline | Ft LVS | Saline | Ft LVS | Saline | Ft LVS |
| Pro-Inflammatory | TNF-α | 1 | 16 | 1 | 17 | 1 | 37 |
| | IL-12 p35 | 2 | 1 | 1 | 1 | 1 | 6 |
| | IL-12 p40 | 1 | 4 | 1 | 2 | 1 | 21 |
| | IL-1β | 1 | 4 | 1 | 6 | 1 | 8 |
| | IL-6 | 1 | 3 | 1 | 3 | 2 | 12 |
| Interferons | IFN-β | 1 | 1 | 3 | 3 | 1 | 2 |
| | IFN-γ | 1 | 155 | 1 | 11 | 1 | 765 |
| Chemokines | IP-10 | 1 | 44 | 1 | 57 | 1 | 133 |
| | KC | 1 | 14 | 1 | 30 | 1 | 69 |
| | MCP-1 | 1 | 21 | 1 | 31 | 1 | 107 |
| | Rantes | 1 | 1 | 1 | 1 | 1 | 3 |
| Anti- | IL-4 | 1 | 1 | 1 | 1 | 1 | 1 |
| Inflammatory | IL-10 | 1 | 1 | 1 | 1 | 1 | 2 |
| Others | iNOS | 1 | 99 | 1 | 70 | 1 | 535 |

These data were confirmed at the level of protein for TNF-α, IL-12p70, IL-6, IFN-γ, KC, MCP-1, Rantes, and IL-10 (Table V). It is noteworthy that levels of IFN-γ and iNOS mRNA were particularly sensitive to FT LVS LPS pretreatment.

TABLE V
Effect of Ft LVS LPS pretreatment of Ft LVS-challenged mice on cytokine levels in sera.

| | | Pretreatment | | | | | |
|---|---|---|---|---|---|---|---|
| | | −2 LPS | −2 LPS | −7 LPS | −7 LPS | −7 Saline | −7 Saline |
| | | Day O | | | | | |
| | | | Ft | | Ft | | |
| | | Saline | LVS | Saline | LVS | Saline | Ft LVS |
| Pro-Inflammatory | TNF-α | * | 29.6 ± 1.3 | * | 21.9 ± 1.9 | * | 78.8 ± 9.3 |
| | IL-12 p70 | * | 20.5 ± 2.3 | * | 0 | * | 35.5 ± 1.8 |
| | IL-6 | * | 106.7 ± 2.8 | * | 110 ± 0.6 | * | 460 ± 55.5 |
| Interferons | IFN-γ | * | 843.3 ± 91.0 | * | 244 ± 5.0 | * | 2406 ± 350.8 |
| Chemokines | KC | 44.3 ± 3.5 | 217 ± 4.0 | 55.7 ± 1.6 | 236.6 ± 9.5 | 43.4 ± 0.4 | 1513.3 ± 155.0 |
| | MCP-1 | 56.5 ± 3.3 | 803.3 ± 84.0 | 32.7 ± 2.1 | 843.3 ± 19.5 | 9.42 ± 2.6 | 1776.7 ± 110.2 |
| Anti- | IL-10 | * | 7.0 ± 1.2 | * | 2.5 ± 2.6 | 0.8 ± 1.3 | 15.9 ± 0.9 |
| Inflammatory | | | | | | | |

*No cytokine detected, concentration below limit of detection

In contrast to Ft LVS, which is F. tularensis subspecies holoarctica, biovar type B, the SHU S4 strain is a much more virulent strain of F. tularensis and belongs to the subspecies tularensis, biovar type A. Two days after groups of 4 wild-type C57BL/6 mice were given an intraperitoneal injection of saline or Ft LVS LPS (100 ng), mice were intraperitoneally challenged with either 10 or 100 CFU of Ft SHU S4. The data suggests that Ft LVS LPS partially protects individuals against the more virulent SHU S4 strain. The only animal that survived the challenge was pretreated with 100 ng Ft LVS LPS. Also the mean times to death were increased in mice pretreated with Ft LVS LPS. Specifically, the mean time of death in animals challenged with 10 CFU of Ft SHU S4 increased from approximately 4.25 days to approximately 6 days, and the mean time of death for animals challenged with 100 CFU rose from approximately 4.5 days to approximately 5.75 days as a result of pretreatment with Ft LVS LPS.

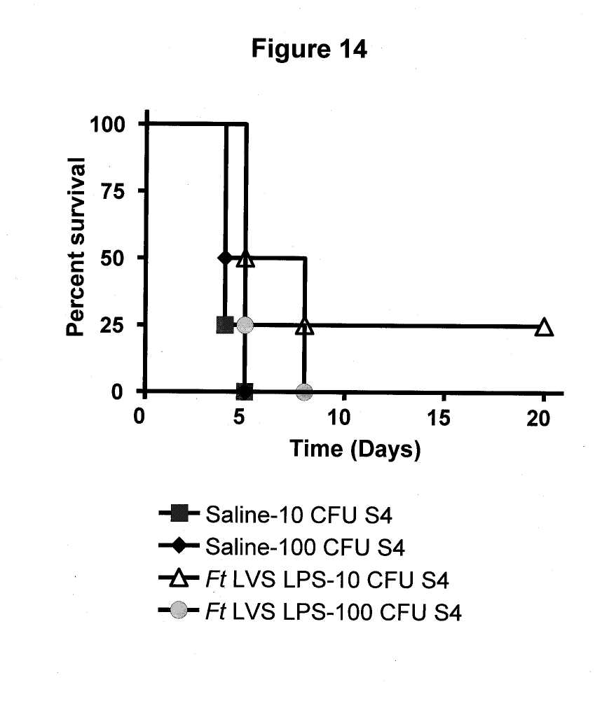

Administering 100 ng Ft LVS LPS, therefore, afforded protection against the SHU4 strain of Ft. (FIG. 14). There was a statistically significant difference in the survival curves (P=0.0310) between the 10 CFU SHU4-challenged animals receiving saline and animals receiving Ft LVS LPS. These results show that Ft LVS LPS mediates protection even against highly virulent strains of Ft.

All references are herein incorporated by reference.
Petersen, J. M. and M. E. Schriefer. 2005. Tularemia: emergence/re-emergence. Vet. Res. 36:455-467.
Elkins, K. L., S. C. Cowley, and C. M. Collazo. 2003. Francisella: a little bug hits the big time. Expert. Rev. Vaccines. 2:735-738.
Sjostedt, A. 2003. Virulence determinants and protective antigens of Francisella tularensis. Curr. Opin. Microbiol. 6:66-71.
Tarnvik, A. 1989. Nature of protective immunity to Francisella tularensis. Rev. Infect. Dis. 11:440-451.
2002. Tularemia—United States, 1990-2000. MMWR Morb. Mortal. Wkly. Rep. 51:181-184.
Oyston, P. C., A. Sjostedt, and R. W. Titball. 2004. Tularaemia: bioterrorism defence renews interest in Francisella tularensis. Nat. Rev. Microbiol. 2:967-978.
Kortepeter, M. G. and G. W. Parker. 1999. Potential biological weapons threats. Emerg. Infect. Dis. 5:523-527.
Elkins, K. L., S. C. Cowley, and C. M. Bosio. 2003. Innate and adaptive immune responses to an intracellular bacterium, Francisella tularensis live vaccine strain. Microbes. Infect. 5:135-142.
Tarnvik, A., M. Eriksson, G. Sandstrom, and A. Sjostedt. 1992. Francisella tularensis—a model for studies of the immune response to intracellular bacteria in man. Immunology 76:349-354.
Eigelsbach, H. T. and C. M. DOWNS. 1961. Prophylactic effectiveness of live and killed tularemia vaccines. I. Production of vaccine and evaluation in the white mouse and guinea pig. J. Immunol. 87:415-425.
SASLAW, S., H. T. EIGELSBACH, H. E. WILSON, J. A. PRIOR, and S. CARHART. 1961. Tularemia vaccine study. I. Intracutaneous challenge. Arch. Intern. Med. 107:689-701.
SASLAW, S., H. T. EIGELSBACH, J. A. PRIOR, H. E. WILSON, and S. CARHART. 1961. Tularemia vaccine study. II. Respiratory challenge. Arch. Intern. Med. 107:702-714.
DIENST, F. T., Jr. 1963. Tularemia: a perusal of three hundred thirty-nine cases. J. La State Med. Soc. 115:114-127.
Elkins, K. L., R. K. Winegar, C. A. Nacy, and A. H. Fortier. 1992. Introduction of Francisella tularensis at skin sites induces resistance to infection and generation of protective immunity. Microb. Pathog. 13:417-421.
Fortier, A. H., M. V. Slayter, R. Ziemba, M. S. Meltzer, and C. A. Nacy. 1991. Live vaccine strain of Francisella tularensis: infection and immunity in mice. Infect. Immun. 59:2922-2928.
Green, M., G. Choules, D. Rogers, and R. W. Titball. 2005. Efficacy of the live attenuated Francisella tularensis vaccine (LVS) in a murine model of disease. Vaccine 23:2680-2686.
Elkins, K. L., T. Rhinehart-Jones, C. A. Nacy, R. K. Winegar, and A. H. Fortier. 1993. T-cell-independent resistance to infection and generation of immunity to Francisella tularensis. Infect. Immun. 61:823-829.
Elkins, K. L., D. A. Leiby, R. K. Winegar, C. A. Nacy, and A. H. Fortier. 1992. Rapid generation of specific protective immunity to Francisella tularensis. Infect. Immun. 60:4571-4577.
Stenmark, S., D. Sunnemark, A. Bucht, and A. Sjostedt. 1999. Rapid local expression of interleukin-12, tumor necrosis factor alpha, and gamma interferon after cutaneous Francisella tularensis infection in tularemia-immune mice. Infect. Immun. 67:1789-1797.
Baskerville, A. and P. Hambleton. 1976. Pathogenesis and pathology of respiratory tularaemia in the rabbit. Br. J. Exp. Pathol. 57:339-347.
Bolger, C. E., C. A. Forestal, J. K. Italo, J. L. Benach, and M. B. Furie. 2005. The live vaccine strain of Francisella tularensis replicates in human and murine macrophages but induces only the human cells to secrete proinflammatory cytokines. J. Leukoc. Biol.
Conlan, J. W. and R. J. North. 1992. Early pathogenesis of infection in the liver with the facultative intracellular bacteria Listeria monocytogenes, Francisella tularensis, and Salmonella typhimurium involves lysis of infected hepatocytes by leukocytes. Infect. Immun. 60:5164-5171.
Conlan, J. W., R. Kuolee, H. Shen, and A. Webb. 2002. Different host defences are required to protect mice from primary systemic vs pulmonary infection with the facultative intracellular bacterial pathogen, Francisella tularensis LVS. Microb. Pathog. 32:127-134.
Forestal, C. A., J. L. Benach, C. Carbonara, J. K. Italo, T. J. Lisinski, and M. B. Furie. 2003. Francisella tularensis selectively induces proinflammatory changes in endothelial cells. J. Immunol. 171:2563-2570.
Moe, J. B., P. G. Canonico, J. L. Stookey, M. C. Powanda, and G. L. Cockerell. 1975. Pathogenesis of tularemia in immune and nonimmune rats. Am. J. Vet. Res. 36:1505-1510.
Schricker, R. L., H. T. Eigelsbach, J. Q. Mitten, and W. C. Hall. 1972. Pathogenesis of tularemia in monkeys aerogenically exposed to Francisella tularensis 425. Infect. Immun. 5:734-744.
Golovliov, I., G. Sandstrom, M. Ericsson, A. Sjostedt, and A. Tarnvik. 1995. Cytokine expression in the liver during the early phase of murine tularemia. Infect. Immun. 63:534-538.
Golovliov, I., K. Kuoppa, A. Sjostedt, A. Tarnvik, and G. Sandstrom. 1996. Cytokine expression in the liver of mice infected with a highly virulent strain of Francisella tularensis. FEMS Immunol. Med. Microbiol. 13:239-244.
Fortier, A. H., S. J. Green, T. Polsinelli, T. R. Jones, R. M. Crawford, D. A. Leiby, K. L. Elkins, M. S. Meltzer, and C. A. Nacy. 1994. Life and death of an intracellular pathogen: Francisella tularensis and the macrophage. Immunol. Ser. 60:349-361.
Elkins, K. L., T. R. Rhinehart-Jones, S. J. Culkin, D. Yee, and R. K. Winegar. 1996. Minimal requirements for murine resistance to infection with Francisella tularensis LVS. Infect. Immun. 64:3288-3293.
Collazo, C. M., A. Sher, A. I. Meierovics, and K. L. Elkins. 2005. Myeloid Differentiation Factor-88(MyD88) is essential for control of primary in vivo Francisella tularensis LVS infection, but not for control of intramacrophage bacterial replication. Infect. Immun.
Lindgren, H., S. Stenmark, W. Chen, A. Tarnvik, and A. Sjostedt. 2004. Distinct roles of reactive nitrogen and oxygen species to control infection with the facultative intracellular bacterium Francisella tularensis. Infect. Immun. 72:7172-7182.

Ancuta, P., T. Pedron, R. Girard, G. Sandstrom, and R. Chaby. 1996. Inability of the Francisella tularensis lipopolysaccharide to mimic or to antagonize the induction of cell activation by endotoxins. Infect. Immun. 64:2041-2046.
Sandstrom, G., A. Sjostedt, T. Johansson, K. Kuoppa, and J. C. Williams. 1992. Immunogenicity and toxicity of lipopolysaccharide from Francisella tularensis LVS. FEMS Microbiol. Immunol. 5:201-210.
Phillips, N. J., B. Schilling, M. K. McLendon, M. A. Apicella, and B. W. Gibson. 2004. Novel modification of lipid A of Francisella tularensis. Infect. Immun. 72:5340-5348.
Vinogradov, E., M. B. Perry, and J. W. Conlan. 2002. Structural analysis of Francisella tularensis lipopolysaccharide. Eur. J. Biochem. 269:6112-6118.
Loppnow, H., H. Brade, I. Durrbaum, C. A. Dinarello, S. Kusumoto, E. T. Rietschel, and H. D. Flad. 1989. IL-1 induction-capacity of defined lipopolysaccharide partial structures. J. Immunol 142:3229-3238.
Fulop, M., R. Manchee, and R. Titball. 1995. Role of lipopolysaccharide and a major outer membrane protein from Francisella tularensis in the induction of immunity against tularemia. Vaccine 13:1220-1225.
Fulop, M., P. Mastroeni, M. Green, and R. W. Titball. 2001. Role of antibody to lipopolysaccharide in protection against low- and high-virulence strains of Francisella tularensis. Vaccine 19:4465-4472.
Dreisbach, V. C., S. Cowley, and K. L. Elkins. 2000. Purified lipopolysaccharide from Francisella tularensis live vaccine strain (LVS) induces protective immunity against LVS infection that requires B cells and gamma interferon. Infect. Immun. 68:1988-1996.
Conlan, J. W., E. Vinogradov, M. A. Monteiro, and M. B. Perry. 2003. Mice intradermally-inoculated with the intact lipopolysaccharide, but not the lipid A or O-chain, from Francisella tularensis LVS rapidly acquire varying degrees of enhanced resistance against systemic or aerogenic challenge with virulent strains of the pathogen. Microb. Pathog. 34:39-45.
Salkowski, C. A., K. Kopydlowski, J. Blanco, M. J. Cody, R. McNally, and S. N. Vogel. 1999. IL-12 is dysregulated in macrophages from IRF-1 and IRF-2 knockout mice. J. Immunol. 163:1529-1536.
McIntire, F. C., H. W. Sievert, G. H. Barlow, R. A. Finley, and A. Y. Lee. 1967. Chemical, physical, biological properties of a lipopolysaccharide from Escherichia coli K-235. Biochemistry 6:2363-2372.
Westphal, O. and K. Jann. 1965. Bacterial Lipopolysaccharides. In Methods in Carbohydrate Chemistry. R. L. Whistler, ed. Academic Press, New York, pp. 83-91.
Manthey, C. and Vogel S. N. 1994. Elimination of trace endotoxin protein from rough chemotype LPS. J Endotoxin Res. 1:84-91.
Cuesta, N., C. A. Salkowski, K. E. Thomas, and S. N. Vogel. 2003. Regulation of lipopolysaccharide sensitivity by IFN regulatory factor-2. J. Immunol. 170:5739-5747.
Azam, P., J. L. Peiffer, J. C. Ourlin, P. A. Bonnet, M. H. Tissier, L. Vian, and I. Fabre. 2005. Qualitative and quantitative evaluation of a local lymph node assay based on ex vivo interleukin-2 production. Toxicology 206:285-298.
Medvedev, A. E. and S. N. Vogel. 2003. Overexpression of CD14, TLR4, and MD-2 in HEK 293T cells does not prevent induction of in vitro endotoxin tolerance. J. Endotoxin. Res. 9:60-64.
Miller, S. I., R. K. Ernst, and M. W. Badger. 2005. LPS, TLR4 and infectious disease diversity. Nat. Rev. Microbiol. 3:36-46.
Miyake, K. 2004. Innate recognition of lipopolysaccharide by Toll-like receptor 4-MD-2. Trends Microbiol. 12:186-192.
Beutler, B., K. Hoebe, X. Du, and R. J. Ulevitch. 2003. How we detect microbes and respond to them: the Toll-like receptors and their transducers. J. Leukoc. Biol. 74:479-485.
Hirschfeld, M., J. J. Weis, V. Toshchakov, C. A. Salkowski, M. J. Cody, D. C. Ward, N. Qureshi, S. M. Michalek, and S. N. Vogel. 2001. Signaling by toll-like receptor 2 and 4 agonists results in differential gene expression in murine macrophages. Infect. Immun. 69:1477-1482.
Werts, C., R. I. Tapping, J. C. Mathison, T. H. Chuang, V. Kravchenko, G. Saint, I, D. A. Haake, P. J. Godowski, F. Hayashi, A. Ozinsky, D. M. Underhill, C. J. Kirschning, H. Wagner, A. Aderem, P. S. Tobias, and R. J. Ulevitch. 2001. Leptospiral lipopolysaccharide activates cells through a TLR2-dependent mechanism. Nat. Immunol. 2:346-352.
Erridge, C., J. Stewart, E. Bennett-Guerrero, T. J. McIntosh, and I. R. Poxton. 2002. The biological activity of a liposomal complete core lipopolysaccharide vaccine. J. Endotoxin. Res. 8:39-46.
Netea, M. G., M. van Deuren, B. J. Kullberg, J. M. Cavaillon, and J. W. Van der Meer. 2002. Does the shape of lipid A determine the interaction of LPS with Toll-like receptors? Trends Immunol. 23:135-139.
Takayama, K., N. Qureshi, B. Beutler, and T. N. Kirkland. 1989. Diphosphoryl lipid A from Rhodopseudomonas sphaeroides ATCC 17023 blocks induction of cachectin in macrophages by lipopolysaccharide. Infect. Immun. 57:1336-1338.
Dobrovolskaia, M. A., A. E. Medvedev, K. E. Thomas, N. Cuesta, V. Toshchakov, T. Ren, M. J. Cody, S. M. Michalek, N. R. Rice, and S. N. Vogel. 2003. Induction of in vitro reprogramming by Toll-like receptor (TLR)2 and TLR4 agonists in murine macrophages: effects of TLR “homotolerance” versus “heterotolerance” on NF-kappa B signaling pathway components. J. Immunol. 170:508-519.
Cole, L. E., K. L. Elkins, S. M. Michalek, N. Qureshi, L. J. Eaton, P. Rallabhandi, N. Cuesta, and S. N. Vogel. 2006. Immunologic Consequences of Francisella tularensis Live Vaccine Strain Infection: Role of the Innate Immune Response in Infection and Immunity. J Immunol 176:6888-6899.
Katz, J., P. Zhang, M. Martin, S. N. Vogel, and S. M. Michalek. 2006. Toll-like receptor 2 is required for inflammatory responses to Francisella tularensis LVS. Infect Immun 74:2809-2816.

What is claimed is:

1. A pharmaceutical composition comprising a pharmaceutically effective amount of purified lipopolysaccharide (LPS) from Francisella tularensis (F. tularensis) and a pharmaceutically acceptable carrier.

2. The composition of claim 1, wherein the purified LPS is purified from Live Vaccine Strain (LVS) of F. tularensis.

3. A method of treating or preventing a bacterial infection in a subject in need treatment thereof, the method comprising administering the pharmaceutical composition of claim 2 to the subject.

4. The method of claim 3, wherein said bacteria causing said bacterial infection is virulent and is not F. tularensis.

5. The method of claim 3, wherein said bacteria causing said bacterial infection is virulent F. tularensis.

6. The method of claims 3, wherein said purified LPS from F. tularensis is administered to said subject prior to exposure to the virulent bacteria causing said bacterial infection.

7. The method of claim 3, wherein the route of administration is intraperitoneal.

8. A method of modulating the immune response in a subject comprising administering the pharmaceutical composition of claim 2.

9. The method of claim 8, wherein the modulation comprises enhancing the immunocompetence in the subject.

10. The method of claim 8, wherein the modulation comprises inhibiting the production of proinflammatory cytokines in the subject.

11. The method of claim 10, wherein the proinflammatory cytokines comprise at least one cytokine selected from the group consisting of interferon gamma (IFN-γ), tumor necrosis factor alpha (TNF-α), interleukin-4 (IL-4) and cytokine-induced neutrophil chemoattractant (KC).

12. The method of claim 11, wherein the route of administration is intraperitoneal.

13. A method of treating a disease state in a subject in need of treatment thereof, said disease state being marked by inflammation, the method comprising administering the pharmaceutical composition of claim 2 to the subject.

14. The method of claim 13, wherein the route of administration is intraperitoneal.

15. A method of screening candidate compounds for their ability to modulate an immune or inflammatory response, the method comprising
a) providing purified lipopolysaccharide (LPS) from Francisella tularensis (F. tularensis) to a control and test cell population, wherein the control and test cell populations have a measurable immune or inflammatory activity;
b) providing a test agent to the test cell population; and
c) determining a difference in the measurable immune or inflammatory activity between the control and test populations,
wherein a difference in the measurable immune or inflammatory activity between the control and test populations is indicative that the test agent may be used to modulate an immune or inflammatory response.

16. The method of claim 18, wherein the control and test cell populations are selected from the group consisting of hepatocytes and macrophage cells.

17. The method of claim 19, wherein the measurable immune or inflammatory activity comprises production of proinflammatory cytokine mRNA species or the corresponding proteins.

18. The method of claim 17, wherein the proinflammatory cytokines comprise at least one cytokine selected from the group consisting of interferon gamma (IFN-γ), tumor necrosis factor alpha (TNF-α), interleukin-4 (IL-4) and cytokine-induced neutrophil chemoattractant (KC).

19. A method of inhibiting F. tularensis-induced proinflammatory cytokines in a subject, the method comprising selectively inhibiting the activation of TLR2, after the subject has been infected with F. tularensis.

20. The method of claim 19, wherein the proinflammatory cytokines comprise at least one cytokine selected from the group consisting of interferon gamma (IFN-γ), tumor necrosis factor alpha (TNF-α), interleukin-4 (IL-4) and cytokine-induced neutrophil chemoattractant (KC).
